(19) United States
(12) Patent Application Publication
(10) Pub. No.: US 2006/0073357 A1
(43) Pub. Date: Apr. 6, 2006

(54) Carbazole compounds and use of such compounds in organic electroluminiscent devices

(76) Inventors: Klemens Brunner, Eindhoven (NL); Margaretha Maria De Kok-Van Breemen, Eindhoven (NL); Bea Maria Wilhelmina Langeveld, Milsbeek (NL); Nicole Maria Matthias Kiggen, Leveroy (NL); Jolanda Johanna Anna Maria Bastiaansen, Helmond (NL); Johannes Willem Hofstraat, Eindhoven (NL); Herbert Friedrich Boerner, Aachen (NL); Hermannus Franciscus Maria Schoo, Eersel (NL)

Correspondence Address: Philips Electronics North America Corporation;Corporate Patent Counsel, P O Box 3001, Briarcliff Manor, NY, 10510-8001, US

(21) Appl. No.: 10/545,205

(22) PCT Filed: Jan. 23, 2004
(86) PCT No.: PCT/IB04/50049

§ 371 Date: Aug. 10, 2005

(30) Foreign Application Priority Data
Feb. 12, 2003 (NL) 1022660

(51) Int. Cl. B32B 19/00

(52) U.S. Cl. 428/690

(57) ABSTRACT

A carbazole compound, polymeric or of low molecular weight, comprises a carbazole multimer unit of formula (I), wherein each carbazole unit may be unsubstituted or substituted with one or more substituents and n is larger than or equal to 2 for use in organic electroluminescent devices. The carbazole compounds provide facile hole-injection from a hole-injecting electrode and have a relatively triplet level enabling highly efficient electroluminescent devices to be obtained if combined with triplet emitter compounds.

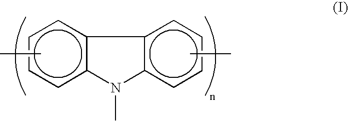

### FIELD OF THE INVENTION

The invention relates to carbazole compounds and the use of such compounds in organic electroluminescent devices.

### BACKGROUND OF THE INVENTION

An electroluminescent (EL) device is a device which emits light when a suitable voltage is impressed on its electrodes. If the electroluminescent device comprises one or more organic compounds which facilitate charge transport and/or light emission it is generally referred to as an organic electroluminescent device. Organic electroluminescent devices are low-voltage devices which can be made to emit any color and are thin, light weight, flexible and/or of large area rendering such devices suitable for display and lighting applications. An organic electroluminescent device may comprise organic compounds of relatively low molecular weight, also referred hereinafter as small molecule electroluminescent devices, or compounds of high molecular weight, hereinafter referred to as polymer electroluminescent devices.

Organic compounds for use in organic electroluminescent devices, both small molecule and polymer, are generally conjugated to facilitate charge transport and/or light emission in the visible range of the electromagnetic spectrum. Although many different conjugated compounds for use in organic electroluminescent devices have hitherto been suggested in the art there is still a need for further conjugated compounds which allow electroluminescent devices to be obtained having desirable properties.

### SUMMARY OF THE INVENTION

Accordingly, it is an object of the invention, inter alia, to provide further conjugated compounds which can be suitably used in organic electroluminescent devices.

In accordance with the invention, this object is achieved by a carbazole compound comprising a carbazole multimer unit of formula (I) 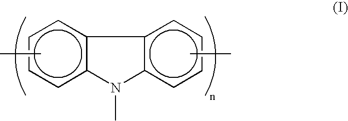
wherein each carbazole unit may be unsubstituted or substituted with one or more substituents and n is larger than or equal to 2.

Preferably, 2≦n≦100,000, more particular, 2≦n≦10,000. More preferably, 2≦n≦10, or n=2, 3, 4 or 5.

The compounds in accordance with the invention can be suitably used, if appropriate in combination with other compounds, to provide red, green or blue light emission. More in particular, when used as such, the compounds in accordance with the invention may be used to provide electroluminescent devices emitting blue light.

Further, the compounds in accordance with the invention may be used to provide an electroluminescent device with a hole-accepting level having an energy comparable to the work-function of high-work function materials such as indium tin oxide. In particular, the energy of the hole-accepting level, expressed in terms of the energy of the highest occupied molecular orbital of the compound in accordance with the invention may be about 5.4 eV or lower.

Still further, the compounds in accordance with the invention may be suitably used as a donor compound for transferring energy, where energy may take the form of excitons and/or charges, to an acceptor compound which has a high quantum yield of light emission. More in particular, such acceptor compounds may be red, green or blue light-emitting compounds. Donor acceptor systems are of particular advantage in multi-color electroluminescent devices. The acceptor is typically present in amounts so small that, if the donor and acceptor are provided as part of a single layer, the processing of the different emissive regions is determined by the donor and therefore essentially identical regardless the color emitted. Further, charge injection and transport is essentially determined by the donor compound and therefore essentially color-independent.

Also, the compounds or more particular the polymers in accordance with the invention may be used to provide electroluminescent devices wherein during operation energy is transferred from the compound in accordance with the invention (the donor) to a red and/or green and/or blue triplet emitter with high efficiency (the acceptor), such use being enabled with compounds or polymers in accordance with the invention having a triplet level sufficiently high in energy to allow energy transfer to a green and/or red light emitting triplet emitter. In particular, the triplet energy may be at least about 20,000 cm⁻¹, 21,000 cm⁻¹ or better 22,000 cm⁻¹.

The carbazole compounds in accordance with the invention may also be used to provide electroluminescent devices having high efficiency.

Excluded carbazole compounds as such are 9,9′-diethyl-3,3′-bi-9H-carbazole, 9,9′-diphenyl-3,3′-bi-9H-carbazole, [3,3′-bi-9H-carbazole]-9,9′-dihexanoic acid, 9,9′-dioctyl-[3,3′-bi-9H-carbazole]-6,6′-dicarbonyl chloride, 9,9′-dioctyl-[2,2′-bi-9H-carbazole]-7,7′-dicarboxylic acid, 9,9′-dioctyl-bis9H-hydroxyethyl) ester, homopolymer, 2,2′-bi-9H-carbazole, 9,9′-diethyl-2,2′-bi-9H-carbazole, and 2,2′-linked carbazole compounds of formula (1) wherein n=4, k≦1, m≦1 for any and all benzene cycles.

Preferred are carbazole compounds comprising a carbazole monomer unit of the formula (Ia) 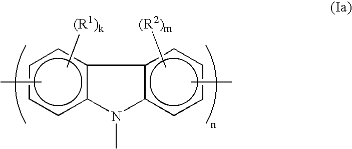
wherein R¹, R², the same or different at each occurrence, a substituent having a total number of non-hydrogen atoms less than 40,
k, m the same or different at each occurrence, 0, 1, 2 or 3;
n an integer equal to or larger than 2.
Suitably, R¹, R² are, the same or different at each occurrence, R⁴, R⁵, R⁷ or R⁸ with
R⁴ is C₁- C₂₀ cyclic or acyclic straight or branched alkyl optionally interrupted one or more times with —O—, —OC(═O)—, —C(═O)O—, —S—, secondary nitrogen, tertiary nitrogen, quaternary nitrogen, —CR⁴⁵═CR⁴⁶—, —C—C—, —C(—O)—, —C(═O)NR⁴⁵—, —NR⁴⁵C(═O)—, —S(═O)—, —S(═O)₂— or —X⁶— and/or substituted one or more times with R⁵, R⁷, R⁸;
R⁵ is C₅-C₃₀ aryl wherein, optionally, one or more of the aromatic carbon atoms are replaced with N, O or S, and, optionally, one or more of the aromatic carbon atoms carry a group R⁴, R⁷, R⁸;
R⁷ is —CN, —CF₃, —CSN, —NH₂, —NO₂, —NCO, —NCS, —OH, —F, —PO₂, —PH₂, —SH, —Cl, —Br, —I;
R⁸ is —C(═O)R⁴⁵, —C(═O)OR⁴⁵, —C(═O)NR⁴⁵R⁴⁶, —NHR⁴⁵, —NR⁴⁵R⁴⁶, —N⁽⁺⁾R⁴⁵R⁴⁶R⁴⁷, —NC(═O)R⁴⁵—, —OR⁴⁵, —OC(═O)R⁴⁵, —SR⁴⁵, —S(═O)R⁴⁵O, —S(═O)₂R⁴⁵;
wherein
R⁴⁵, R⁴⁶, R⁴⁷, the same or different, H, R⁴ or R⁵;
X⁶ is C₄-C₃₀ arylene wherein, optionally, one or more of the aromatic carbon atoms are replaced with N, O or S and, optionally, one or more of the aromatic carbon atoms carry a group R⁴, R⁷, R⁸.

In a preferred embodiment of the invention, one or more consecutive carbazole units are connected to one another via their position, in other words the carbazole monomer unit includes a 2,2′-bicarbazole-diyl unit such as a 2,2′-bicarbazole-7,7′-diyl unit of formula (II), 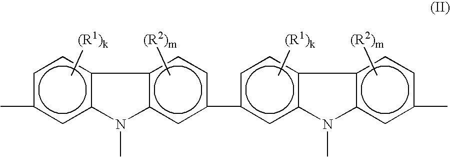
wherein the indices and symbols have the meaning as hereinabove. It is preferred that each pair of consecutive carbazole units of the carbazole multimer is a 2,2′-bicarbazole-diyl unit or more particular a 2,2′-bicarbazole-7,7′-diyl unit.

In a more preferred embodiment of the invention, the carbazole monomer unit includes a 3,3′-bicarbazole-diyl unit such as a 3,3′-bicarbazole-6,6′-diyl unit of formula (III) 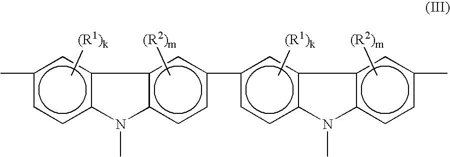
wherein the indices and symbols have the meaning as hereinabove. It is preferred that each pair of consecutive carbazole units of the carbazole multimer is a 3,3 ′-bicarbazole-diyl unit or more particular 3,3′-bicarbazole-6,6′-diyl unit.

Compounds comprising 3,3′-bicarbazole-6,6′-diyl units provide electroluminescent devices having high efficiency.

In formula (I), the total number of non-hydrogen atoms of each substituent R¹ and R² is preferably less than 20, even more particular, less than or equal to 12, still more particular less than or equal to 6.

Further, k and m of each individual carbazole unit satisfy k+m≦2, preferably k+m≦1 or k+m=0.

More in particular, the total number of non-hydrogen carbon atoms of the substituents R¹ and R² on each individual carbazole-diyl unit is less than 40, preferably less than 20, or more preferable less than 10.

A small number of non-hydrogen atoms in the substituents R¹ and R² is particularly advantageous if the compounds in accordance with the invention are used in an electroluminescent device as the substituents typically do not participate in charge injection, transport or light emission.

R⁴ is more particularly defined as C₁-C₆ (hetero)alkyl, such as methyl, ethyl, i-propyl, n-propyl, butyl, cyclopentyl and cyclohexyl.

Rs is more particularly defined as C₄-C₁₈ (hetero)aryl, such as substituted or unsubstituted furanyl, thienyl, and pyrrolyl, cyclopentadienyl, butadienyl, unsubstituted or substituted phenyl, such as tolyl, xylyl, trimethylphenyl, methoxyphenyl, dimethoxyphenyl, butoxyphenyl, dibutoxyphenyl, pentoxyphenyl, or heterophenyl, such as pyrimidyl, pyridinyl and pyrazinyl, biphenyl, and naphthyl.

R⁷ is preferably F, CF₃ or NH₂.

R⁸ is preferably C₁-C₆ (hetero)alkoxy such as methoxy, ethoxy, n-propoxy, i-propoxy, butoxy, cyclopentyloxy, cyclohexyloxy. R⁸ may also be C₄-C₁₈ aryloxy where aryl is R⁵ as more particularly defined hereinabove. R⁸ may also be C₁-C₆ monoalkylaminc, such as methylamino, C₆-C₁₂ arylamino, such phenylamino, and naphthylamino, C₁-C₆ dialkylamino, such as diethylamino, or C₆-C₁₂ diarylamino such as diphenyl amino. X⁶ is preferably unsubstituted or substituted phenylene.

The carbazole compound in accordance with the invention, carbazole polymers as well as carbazole compounds of low molecular weight, may include conjugated units other than the carbazole multimer unit of formulas (I), (Ia) (II) or (I). Such an additional conjugated unit is attached to a dangling bond on the terminal carbazole units of the multimer. The one or more additional conjugated units may be linked via a saturated unit such that no conjugated path from the additional conjugated unit to the carbazole multimer unit exists but preferably the additional conjugated unit is linked directly to the carbazole multimer unit to establish through-conjugated system. Preferably, the carbazole compound is of the formula (IV) 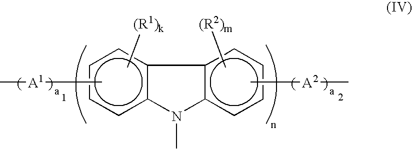
wherein A¹ and A² are, the same or different at each occurrence,
—CR¹¹—CR¹²— with R¹¹ and R¹² the same or different, H, methyl, CF₃, F, CN, phenyl, —N═N—, —CR¹¹═N—, —NR⁴⁵—, —O—, —S—, —C≡C— or C₄-C₅₀ aryl wherein optionally one or more aromatic carbon atoms is substituted with O, S or N and optionally or more carbon atoms is substituted with R¹. More particular, A¹ and A² are, the same or different at each occurrence, unsubstituted or substituted phenylene, pyrimidinylene, pyridinylene, pyrazinylene, thienylene, furandiyl, pyrrolediyl, naphthalenediyl, thiadiazol-diyl, oxadiazole-diyl, anthracene-diyl, phenanthrene-diyl, benzothiadiazole, 6,7-dihydrophenanthrene-diyl, pyrene-diyl, 5,6,9,10-etrahydropyrene-diyl. Indices a₁ and a₂, the same or different, a₁+a₂=1 to 10, preferably 1 to 5, such as 1, 2, 3 or 4.

In particular if the carbazole compound is for use in an organic electroluminescent device, the additional conjugated unit is selected to be a unit known to be of use for charge injection (CI), charge transport (CT) and/or light emission (LE) in such devices where charge-transport and/or light emission relates to electron injection (EI) and/or transport (ET) and/or hole-injecting (HI) and/or transport (HT) units, hole blocking (HB) or electron blocking (EB), charge and/or exciton trapping units, exciton blocking units, exciton migration units, singlet light-emission (SE) and/or triplet light-emission (TE).

Generally, saturated atoms provide charge and/or exciton blocking functionality whereas the conjugated atoms may provide any of the functionalities mentioned above.

Preferred carbazole compounds of formula (IV) are those wherein a₁=a₂=1 or a₁+a₂=1 and A¹ and A², the same or different, unsubstituted or substituted phenylene, such as 1,4 phenylene or 1,3-phenylene, 1,4 phenylenevinylene or 1,3-phenylenevinylene, biphenylene, terphenylene and phenylenevinylenephenylene, biphenylenevinylene, naphthylene, in particular 2,6-naphthylene, 2,7-naphthylene, phenanthrene-diyl, 5,6-dihydrophenanthrene-diyl, pyrene-diyl and anthracene-diyl wherein each of these (hetero)aryls may be unsubstituted or substituted with one or more substituents R¹. Thienylenevinylene, ethylenedioxy-thienylene, phenylene-N-, perylene-diyl, acridine-diyls and coumarine-diyls may also be used. Of particular use are those wherein (A¹)ₐ₁ and (A²)ₐ₂ is, the same or different, of the formula (V), (VI), (VII) or (VIII) 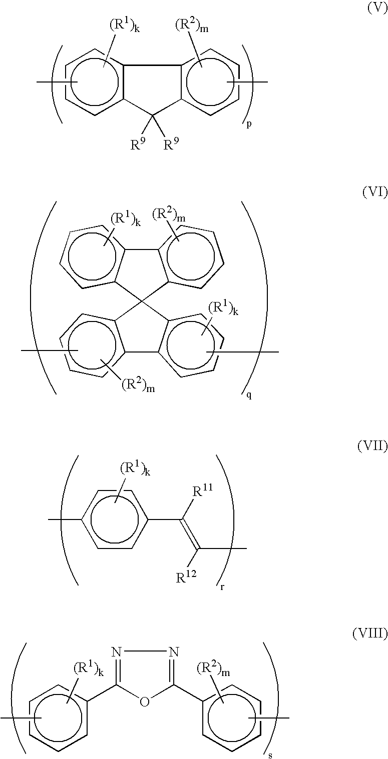
wherein
R⁹ is, the same or different, R¹ or R², p, q, r, and s, the same or different, 1 to 10, in particular, 2 to 5. Indices k and m and symbols R¹, R², R¹¹ and R¹² have the (preferred) meaning as hereinabove. In formula (V), R¹¹ and R¹² are preferably H or CH₃ or phenyl or F or CN, more preferably H. To facilitate synthesis, in formulas (V) and (VI), a fluorene-2,7-diyl unit is preferred. Fluorene-3,6-diyl units are preferred to obtain highenergy triplet levels. Preferably, R⁹ is substituted or unsubstituted phenyl, in particular C₁-C₁₀ alkoxy or alkyl substituted, or C₁-C₁₀ alkyl such as methyl, ethyl, dimethylhexyl.

Preferably, the carbazole compound includes, along with the carbazole multimer, an electron injecting and/or transport repeating unit. Oxadiazole heterocycles and metal complexes of 8-hydroxyquinoline or 8-quinolinol are known to provide such electron injecting and/or transport capability. Preferred is oxadiazole as compounds comprising such heterocycles have a high triplet energy. Particularly preferred is a unit according to formula (VIII). A high triplet energy level is advantageous to highly efficient electroluminescent devices if the carbazole compound is combined with a triplet emitter in particular a green triplet emitter as may facilitate exciton transfer to and/or avoid back-transfer from such triplet emitter.

The unit A¹ and/or A² may also be a unit including a triplet emitting chromophore. Preferred are triplet emitting chromophores which have a large spin-orbit coupling. Generally, spin-orbit coupling increases when a heavy atom is included such as Br, Ru, Rh, Pd, In, I, Hf, Ta, Os, Ir, Pt, Au, Hg, Ti, Pb, Zn and Bi or a rare earth metal such as La, Pr, Nd, Eu, Gd, Tb, Dy, Ho, Er and Tm. Whereas, Br and I may be conveniently introduced as covalently bonded substituents, the other elements may be suitably included in the form of a corresponding ion complexed with ligands, where the ligands are organic moieties which are covalently bonded to the carbazole mulitmer. Such triplet emitting complexes are well known in the art for their pure light emission and high triplet emission efficiency and include in particular porphirine and phtalocyanine complexes of Pt and Ir.

Suitable triplet emitters include but are not limited to those disclosed in U.S. Pat. No. 6,303,238, U.S. Pat. No. 6,310,360, WO00/70655, WO01/41512 and WO 01/39234. Further, Lamanski et al in Inorg. Chem. 40 (2001, page 1704 and Lamanski et al in J. Am. Chem. Soc. 123 (2001), page 4304 disclose the orange-red emitter, iridium(III) bis(2-phenylquinolyl-N,C^{2′}) acetylacetonate, the red emitter Iridium(III) bis(2-(2′-benzothienyl)pyridinato-N,C^{3′}) (acetylacetonate), Iridium(III) bis(2-(2′-thienyl)pyridinato-N,C^{3′}) (acetyl acetonate), Iridium(III) bis(2,4-diphenyloxazolato-1,3-N,C^{2′}) (acetyl acetonate), Iridium(II) bis(3-(2-benzothiazolyl)-7-(diethylamino)-2H-1-benzopyran-2-onato-N′,C⁴) (acetyl acetonate), Iridium(III) bis(2-(2-naphthyl)benzothiazolato-N,C^{2′}) (acetyl acetonate) and Iridium(III) bis(2-phenyl oxazolinato-N,C²′(acetyl acetonate). Commercially available 2,3,7,8,12,13,17,18-Octaethyl-21H,23H-porphyrine platinum (II) may also be used.

Of particular interest are complexes of the formula M³⁺CL⁻₃ UL wherein M=Eu or Tb and CL⁻ is a negatively charged ligand to compensate the ion's charge such as the conjugate base of (2Z)-3-hydroxy-1,3-diphenylprop-2-en-1-one or (4Z)-5-hydroxy-2,2,6,6-tetramethylhept-4-en-3-one or (3Z)-1,1,1-trifluoro-4-hydroxy-4-(2-thienyl)but-3-en-2-one and UL is an uncharged ligand such as 4,7-diphenyl-1,10-phenanthroline, 1,10-henanthroline or 2,2′-bipyridine or combinations thereof.

While not wishing to be bound by any theory, the inventors believe the highly efficient EL devices obtainable with carbazole compounds in accordance with the invention, if combined with triplet emitters, to be due to a combination of a highest occupied molecular orbital having an energy comparable to poly-phenylene-vinylenes achieving facile hole injection and of a triplet level which is high in energy, in particular an energy corresponding to at least green if not blue photons. Such a high triplet would allow triplet excitons formed by recombination of injected holes and electrons to be harvested while preventing back-transfer of excitons residing on the triplet emitter to the carbazole compound. Organic conjugated compounds per se, such as the carbazole compounds in accordance with the invention are generally believed to have a very low efficiency of triplet light emission as the transition involved is a forbidden transition.

The carbazole compound may be a low molecular weight carbazole compound, in particular one suitable for use in a small molecule organic electroluminescent device. Typically, the low molecular weight compound is of the formula (IX) 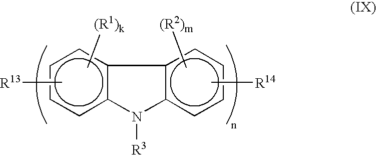
wherein R¹³ and R¹⁴ are, the same or different, R¹, R², R¹-(A¹)ₐ₁ or R²-(A²)ₐ₂, and R³ is an alkyl, heteroalkyl, aryl or heteroaryl substituent having not more than 40 non-hydrogen atoms, with R¹, R², A¹ and A² and the indices a₁, a₂, k, m and n have the (preferred) meaning as hereinabove.

Preferred is n=2 or 3 or 4, k=m=0 at each occurrence, R¹³′R¹⁴═H, and R³ C₁-C₂₂ branched or unbranched alkyl, such as methyl, ethyl, i-propyl, n-propyl, butyl, cyclopentyl, cyclohexyl, pentyl, heptyl, octyl, dimethyloctyl, C₁-C₂₂ branched or unbranched alkoxy such as methoxy, ethoxy, n-propoxy, i-propoxy, butoxy, cyclopentyloxy, cyclohexyloxy, pentyloxy, heptyloxy, octyloxy, decyloxy, substituted or substituted C₄-C₁₈ (hetero)aryl, such as substituted or unsubstituted furanyl, thienyl, and pyrrolyl, cyclopentadienyl, butadienyl, unsubstituted or substituted phenyl, such as tolyl, xylyl, trimethylphenyl, methoxyphenyl, dimethoxyphenyl, butoxyphenyl, dibutoxyphenyl, pentoxyphenyl, or heterophenyl, such as pyrimidyl, pyridinyl and pyrazinyl, biphenyl, and naphthyl.

Also preferred is R¹³═R¹-(A¹)ₐ₁ and R¹⁴═R²-(A²)ₐ₂ with -(A¹)ₐ₁ and -(A²)ₐ₂ a unit of the formula (V), (VI), (VII) and (VI) where the symbols and indices have the meaning described hereinabove.

In an preferred embodiment of the carbazole compound in accordance with the invention, the carbazole compound is a carbazole polymer. In the context of the invention, the term “polymer” includes “oligomer”, “homopolymer” “copolymer”, “terpolymer”, “quaterpolymer” and higher homologues.

In particular, the carbazole compound is a polymer having a repeating unit comprising a carbazole multimer unit of the formula (X) 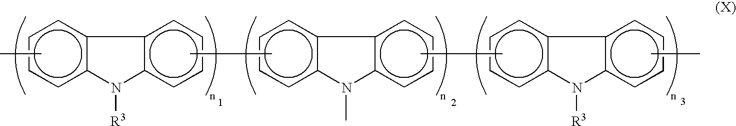
wherein each carbazole unit may be unsubstituted or substituted with one or more substituents and R³ is, the same or different at each occurrence, an alkyl, heteroalkyl, aryl or heteroaryl substituent having not more than 40 non-hydrogen atoms;
n₁ and n₃ are whole numbers including 0, n₂ is 0 or 1 and (n₁+n₂+n₃)≧2.

A carbazole polymer may contain chains having 2 to 100,000 repeating units more particular about 5 to 10,000, more particular about 10 to 1000.

In particular the polymer comprises a unit of the formula (Xa) 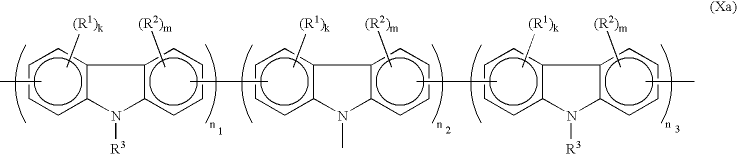
wherein R¹ and R² and the indices k and m have the (preferred) meaning as defined hereinabove.

The carbazole polymer in accordance with the invention may be a linear chain or a cross-linked polymer, the linear polymer more particular a side-chain polymer including the unit of formula (X) as a side group or a main-chain unit.

A side-chain polymer typically includes a carbazole multimer of formula (XI) 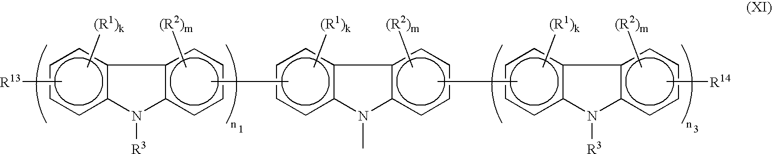
wherein (n₁+n₃)≧1, more particularly (n₁+n₃)=1, 2 or 3, preferably 1 or 2. The side-group is connected to the main chain of the polymer via a nitrogen atom of the carbazole multimer unit. The groups R¹, R², R³, R¹³ and R¹⁴ as well as the indices k and m have the (preferred) meaning as defined with respect to formula (IX).

Alternatively, the carbazole polymer is a side-chain polymer having a repeating unit comprising carbazole multimer of formula (XII) 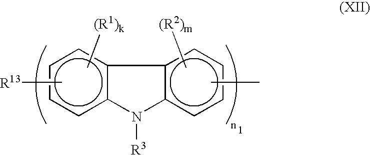
wherein n₁≧2, more particularly n₁ is 2 to 10, preferably 2 or 3. The groups R¹, R², R³ and R¹³ as well as the indices k and m have the (preferred) meaning as defined with respect to formula (XI).

The side-chain groups according to formula (XI) and (XII) may be linked to main chains well known in the art as such, such main chains including those obtained by radical polymerizing one or more vinylene, styrene, proprene or (meth)acrylate monomers functionalized with the side-groups according to the formulas (XI) or (XII). Main chains obtained by condensation polymerization may also be used such as polyesters, polycarbonates, polyamides, polyimides and polyethers.

Preferably, the carbazole multimer unit is part of the main chain of a linear chain polymer in which case the polymer has a repeating unit which may comprise a carbazole multimer of the formula (X) or (Xa) wherein n₃=0, n₂=0 and n₁≧1, preferably n₁ is 1 to 10 or more preferably 1 or 2. A preferred main chain carbazole polymer is one having a repeating unit comprising a carbazole multimer unit of the formula (XIII) 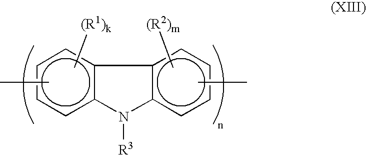
wherein the groups R¹, R² and R³ as well as the indices k, m and n have the (preferred) meaning as defined hereinabove.

The carbazole polymer, side-chain or main chain, may be a homopolymer but may also include a plurality of distinct repeating units to provide copolymers or higher homologues. Main chain copolymers and the like may include additional repeating units which may be saturated, meaning comprising one or more saturated (carbon) atoms such that adjacent conjugated repeating units are not connected by unsaturated atoms, particular examples of which include yet are not limited to repeating units occurring in polyesters, polyethers, polyolefines, poly(meth)acrylates, polyisocyanates, polystyrenes, polyamides, polyvinylacetates and polyimides.

Preferably, however, in order to improve the stress lifetime of the carbazole polymers when used in electroluminescent devices and/or to modify the charge transport and/or light emissive properties of the carbazole polymers in accordance with the invention, the further repeating units are conjugated units which establish a conjugation path between repeating units adjacent thereto. In particular the repeating unit of formula (XIII) is connected to a conjugated repeating unit (A¹)ₐ₁ on the one side and a conjugated repeated unit (A²)ₐ₂ on the other side where A¹, A², a1 and a2 have the (preferred) meaning as defined hereinabove with respect to formula (IV).

The carbazole compounds, both low molecular weight and polymer, as well as monomers from which monomers may be obtained, are available using standard synthetic methods known in the art per se from eg standard works on organic synthesis such as Houben-Weyl, Methoden der Organischen Chemie, Georg-Thieme-Verlag, Stuttgart Carbazole multimer units may be obtained by coupling together suitably functionalized carbazole sub-units using known coupling reactions.

A first known coupling reaction is oxidative coupling of a carbazole derivative by means of an oxidizing agent such as FeCl₃ (see, inter alia, P. Kovacic, N. B. Jones, Chem. Ber. 1987, 87, 357 to 379; M. Weda, T. Abe, H. Awano, Macromolecules 1992, 25, 5125) or electrochemically (see, for example, N. Saito, T. Kanbara, T. Sato, T. Yamamoto, Polym. Bull. 1993, 30, 285).

Other coupling reactions use as starting compounds 2- or 3-monofunctionalized carbazole monomer to obtain 2,2′- or 3,3′-bicarbazoles respectively or where multimers larger than bicarbazoles are desired 2,7- or 3,6-difunctionalized carbazole monomers may be used. Obviously, similarly functionalized carbazole multimers can also be used to make larger multimers or even polymers. Monohalogenated or dihalogenated carbazole monomer or sub-units can be coupled using copper/triphenylphosphine (see, for example, G. W. Ebert, R. D. Rieke, J. Org. Chem. 1988, 53, 44829 or nickel/triphenylphosphine catalysis (see, for example, H. Matsumoto, S. Inaba, R. D. Rieke, J. Org. Chem. 1983, 48, 840).

Diboronic acids of carbazole derivatives and dihalides of carbazole derivatives or mixed monoboronic acid monohalide carbazole derivatives can be coupled reactions using palladium catalysis (see, for example, M. Miyaura, T. Yanagi, A. Suzuki, Synth. Commun. 1981, 11, 513; R. B. Miller, S. Dugar, Organometallics 1984, 3, 1261). Also, mono or difunctionalized stannane carbazoles, can be coupled using palladium catalysis as, for example, indicated in J. K. Stille, Angew. Chem. Int. Ed. Engl. 1986, 25, 508. In addition, dibromo or, where appropriate, monobromo functionalized carbazoles can be converted into the corresponding lithio or Grignard compounds which are then coupled with another dibromo or monobromo carbazole derivatives by means of CuCl₂ (see, for example, G. Wittig, G. Klar, Liebigs Ann. Chem. 1967, 704, 91; H. A. Stabb, F. Bunny, Chem. Ber. 1967, 100, 293; T. Kaufmann, Angew. Chem. 1974, 86, 321 to 354) or by electron transfer of unsaturated 1,4-dihalo compounds (see, for example, S. K. Taylor, S. G. Bennett, K. J. Harz, L. K. Lashley, J. Org. Chem. 1981, 46, 2190).

The coupling reactions may be used to obtain small molecular weight or polymeric carbazole compounds in accordance with the invention. The chain length can be adjusted conveniently by varying the ratio of mono-functionalized to di-functionalized carbazole derivative.

The above-mentioned coupling reactions can also be used to introduce (conjugated) units, in particular cyclic or heterocyclic conjugated units, other than carbazole multimer units in the carbazole compound in accordance with the invention by using suitably mono-functionalized or di-functionalized starting compounds. In particular, the units A¹ and/or A² as defined hereinabove with reference to formula IV, more in particular the fluorene units according to formulas V or VI, can be introduced in this manner. Mono-functionalized or di-functionalized phenyl end-capped phenylenevinylene units can be used to introduce units of formula VII. An oxadiazole unit may be introduced by using 2,5-dihalogenated oxadiazole or more particularly an oxadiazole unit of formula VIII using a corresponding mono- or di-phenyl substituted starting compound.

Copolymers and higher homologues, and low molecular weight compounds comprising a plurality of distinct units may be obtained by jointly coupling corresponding mono-functionalized and/or di-functionalized starting compounds.

In a further aspect, the invention relates to the combination of a carbazole compound in accordance with the invention and a light-emissive compound adapted to be capable of accepting energy from the carbazole compound.

In use, the combination in accordance with the invention picks up energy when exposed to a suitable voltage or radiation which energy is then released at least partially by emission of a photon of light from the light-emissive compound. Energy may be provided in the form of holes, electrons and/or photons. A typical route along which the picking up and release of energy may occur is injection of holes and electrons onto the carbazole compound, formation of an exciton on the carbazole compound by recombination of a hole and electron, transfer of the exciton to the emissive compound and decay of the exciton residing on the emissive compound under emission of a photon. Alternatively, the exciton residing on the carbazole compound may be formed by absorbing a photon of radiation. Instead of an exciton, a hole or electron may be transferred to the light-emissive compound which hole or electron then forms an exciton with an electron or hole respectively already present on the light-emissive compound. In all routes it is desired that the carbazole compound serves as the donor of energy and the light-emissive compound as the acceptor.

Light emission with high efficiency requires a light-emissive compound with quantum efficiency for light emission such compounds are well known in the art. Furthermore, efficient excitonic transfer requires the donor energy level to have a higher preferably slightly higher (about 1 to 5 times kT) energy than the acceptor level. This condition is met if there is spectral overlap of the photo-absorption spectrum of the acceptor and the photo-emission spectrum of the donor. If a hole is to efficiently transferred the hole acceptor level is to be higher in energy than the hole donor level (which may be easily established by measuring the oxidation potential electrochemically). If an electron is to efficiently transferred the electron acceptor level is to be lower in energy than the electron donor level (which may be easily established by measuring the reduction potential electrochemically).

Although in principle any weight ratio of donor and acceptor may be used, the acceptor is typically used in relatively small amounts, donor to acceptor weight or molar ratio typically being about 0.1 to 25, in particular, about 0.5 to 15 or preferably 1 to 10.

The combination in accordance with the invention is of particular advantage in multi-color electroluminescent devices. The acceptor being typically present in amounts so small that, if the donor and acceptor are provided as part of a single layer, the processing of the different emissive regions is determined by the donor and therefore essentially identical regardless the color emitted. Further, charge injection and transport processes are essentially determined by the donor compound and therefore essentially color-independent.

The light-emissive compound may be a singlet-emitter compound also referred to as fluorescent compound or a triplet-emitter compound also referred to as phosphorescent compound distinction between the two is (easily made by the lifetime of the excited state associated with the emission, singlet emissions having a typical lifetime in the nanosecond range and triplet emission having a typical lifetime of at least a value in the microsecond range.

In a preferred embodiment, the light-emissive compound is a triplet emitter compound.

If combined with a triplet emitter compound, light-emission of red, green or may be even blue light as the case may be is emitted with high efficiency in particular if used in an electroluminescent device.

The combination (singlet or triplet) in accordance with the invention may be dispersed in a liquid or may be used in the sold state such as part of a (thin) layer of for example an electroluminescent device.

Triplet emitters compounds which may be suitably used are well known in the art as such and include disclosed in U.S. Pat. No. 6,303,238, U.S. Pat. No. 6,310,360, WO00/70655, WO01/41512 and WO 01/39234. Further, Lamanski et al in Inorg. Chem. 40 (2001, page 1704 and Lamanski et al in J. Am. Chem. Soc. 123 (2001), page 4304.

The triplet acceptor/emitter need only be present in moderate amounts, in particular the ratio of triplet donor to triplet acceptor/emitter level, in weight by weight, is typically 0.1 to 25, more particularly 0.5 to 15. Preferably, the ratio is between about 1 to 10.

The carbazole compound and the light-emissive compound may be combined in a variety of ways.

For example, they may be integrated into a single compound comprising carbazole multimer units and light-emissive unit(s) or may be provided as separate distinct compounds.

If combined in one and the same compound, the one and same compound may be a polymer or a compound of low molecular weight. In the context of the invention, a compound is considered to be of low molecular weight if it can be deposited by means of a vacuum deposition method. The light-emissive unit (triplet emitter unit) may be inserted as a repeating unit of the main chain polymer or may be appended as a side-group. Appendage of the side-group to the main chain may proceed via one or more saturated atoms or may proceed via unsaturated to establish a conjugation path to the main chain. Combination in one and the same compound may be achieved synthetically by means of mono- or, where appropriate, di-functionalizing the light-emissive compound mentioned hereinabove such that the light-emissive compound so functionalized can be coupled to the carbazole multimer unit using the coupling reactions mentioned hereinabove. Combination in one and the same compound has the advantage that the donor and acceptor/emitter units can be brought into a fixed orientation and in close proximity to one another. Also, since only one compound is involved, migration of one compound relative to the other of the combination is prevented. On the down side, combination in one compound requires a more elaborate synthetic effort.

Alternatively, the light-emissive (triplet) compound and the carbazole compound may be provided as separate distinct compounds. They may be both compounds of low molecular weight. A layer comprising such a combination may be deposited by means of a vacuum deposition method or a wet deposition method, such as spin-coating or ink-jet printing, where, if convenient, a (polymeric) binder is added to achieve good film-forming properties. A light-emissive (triplet) compound may also be a polymer combined with a carbazole of low molecular weight. An attractive combination is one wherein the carbazole is of high molecular weight and the light-emissive (triplet) compound is of low molecular weight. The carbazole and light-emissive (triplet) compound may also be both polymers which may be selected to form a single phase or phase-separated polymer blend. Layers comprising combinations including polymer are conveniently formed by means of a wet deposition method, such as spin-coating or ink-jet printing after having been formulated appropriately using solvents and, optionally, other agents which modify the rheologic properties.

Layers comprising carbazole and light-emissive (triplet) compound combinations in accordance with the invention are preferably thin, say 1 nm to 500 μm or more particular 10 nm to 10 μm still more particular 20 nm to 1 μm. Preferably the thickness is about 10 nm to 300 nm.

The carbazole and light-emissive (triplet) compound may be part of one and the same layer, also referred to in the art as a host-guest system. As the light-emissive (triplet) compound is generally present in an amount smaller than the carbazole, the carbazole would normally be the host.

The carbazole and light-emissive (triplet) compound may also be each part of a distinct layer. In order to achieve efficient exciton transfer from donor to emitter across the interface between the layers, the layers are to be in close proximity, preferably therefore the layers are in direct contact.

The carbazole compound may be combined with more than one emitter compound where each emitter compound emits light of a different color. All but one of such emitter compounds may be a singlet emitter compound but preferably more or all emitter compounds are triplet emitter compounds. By varying the relative amounts of each such triplet emitter a range of colors can be obtained. For example, white light emission can be obtained if a red, blue and green emitter or a blue and yellow emitter are combined.

Compositions and bodies, in particular layers, comprising combinations of carbazole and light-emissive (triplet) compound in accordance with the invention may comprise further components. If a layer or composition is used in an electroluminescent device such further components include compounds for modifying the charge transport and exciton transfer properties or the color of light emission of the layer or composition. As the carbazole multimer units provide hole-injection and transport functionality and the emitter compound light emission functionality, a preferred further component is an electron-injecting transport compound. Such compounds are well known in the art as such. Anti-oxidants and agents for improving film formation may also be used if appropriate.

The carbazole compounds of the present invention whether or not combined with light-emissive (triplet) compounds have many interesting applications among them photo-voltaic devices and polymer electronics. Diagnostics of biological samples is another. A particular attractive application is organic electroluminescent devices.

The invention also relates to a combination of a charge-transporting conjugated compound having a triplet level with an energy of about 21,000 cm−1 or higher and a triplet emitter compound having an emission level with an energy of about 21,000 cm−1 or lower. Better the triplet energy level is about 22,000 cm−1 or higher and the emission energy level about 22,000 cm−1 or lower. To prevent back-transfer is the emission level is preferably somewhat lower in energy, say 10 to 40 nm. The charge-transporting conjugated compound may be of low molecular weight but is preferably a polymer. Preferably, but not necessarily, the polymer or low molecular weight compound includes carbazole multimer.

Efficient hole-injection is achieved if the highest occupied molecular orbital of the charge-transporting conjugated compound has an energy of less than or equal to about 5.4 eV or better less than or equal to about 5.3 eV.

Lowest energy triplet level of the charge-transporting conjugated compound corresponds to the peak intensity wavelength of the phosphorescence emission band of lowest energy with the proviso that if such a band shows several peaks due to %,ibronic progression the highest energy peak among these several peaks is taken to correspond to the lowest triplet level.

The (preferred) combination of a triplet emitter having an emission level selected to have an energy at about the same or slightly below the lowest triplet level of a charge-transporting conjugated compound provide light emission with high efficiency for example if used in an EL device. High efficiency is at least obtained for triplet emitters emitting red, orange, yellow and green light and possibly blue light if the lowest triplet energy is at least 21,000 cm−1 or better 22,000 cm−1. The high efficiency is believed to be a consequence of the relatively high energy of the triplet level which enables transfer of triplet excitons formed on the conjugated compound to the triplet emitter thus making such triplet excitons available for light emission from the triplet emitter. Such triplet excitons would otherwise decay radiation-less. Also, the high energy triplet level prevents back-transfer of (single or triplet) excitons formed on the triplet emitter to the conjugated compound thus eliminating an important radiation-less pathway.

In a further aspect, the invention relates to an electroluminescent device including a carbazole compound in accordance with the invention or a combination of a triplet donor carbazole compound and a triplet emitter compound.

The presence of the carbazole multimer units in the electroluminescent device provides the organic electroluminescent device with good hole-injection properties and if combined with light-emissive (triplet) emissive compounds devices capable of emitting red, yellow or green or blue light with high efficiency.

The carbazole multimer units provide the carbazole compound with a HOMO having an energy comparable to the work function of conventional hole-injecting electrodes such as indiumtinoxide (ITO).

The electroluminescent devices including the carbazole compound or the combination of such carbazole compound and triplet emitter compound may be of a conventional nature.

In its simplest form, the electroluminescent device comprises an organic electroluminescent layer comprising carbazole compound or combination in accordance with the invention dispersed between a hole-injecting and electron-injecting electrode.

Other device configurations include HIE/HTL/LEL/EIE, HIE/LEL/ETL/EIE, HIE/HTL/LEL/ETL/EIE, HIE/LEL/HBL/EIE, HIE/EBL/LEL/EIE, HIE/HTL/LEL/HBL/EIE, HIE/HTL/EBL/LEL/EIE, HIE/LEL/HBL/ETL/EIE, HIE/HTL/LEL/HBL/ETL/EIE, HIE/HTL/HBL/LEL/ETL/EIE or HIE/HTL/HBL/LEL/EIE wherein HIE means hole-injection electrode, EIE electron-injecting electrode, HTL hole-transport and/or hole-injection layer, ETL electron-transport and/or injection layer, LEL light-emission layer, HBL hole-blocking layer and EBL electron-blocking layer. Such layers are known in the art as such and may be suitably used in the electroluminescent device in accordance with the invention.

The electroluminescent device may be a light emitting diode comprising a high-work function hole-injecting electrode such as Pd, Pt, Au, Ag, Al, and ITO, and a low work function electron-injecting electrode including low work function metal such as Al, Ca, Ba, Sm, Yb, Li, and Mg. Alternatively the electroluminescent device may be light-emitting electrochemical cell which may be provided with high-work function electron and hole-injecting electrodes.

The electroluminescent device generally comprises a substrate. Suitable substrate materials include glass, ceramics, metals and synthetic resins or combinations of such materials. Typically, since organic electroluminescent devices are sensitive to oxygen and water the substrate serves as a barrier for ingress for water and oxygen. In the case of synthetic resins barrier properties may be improved by including barrier layer(s) of glass, ceramic or metal. Although in particular light emitting chemical cells may have hole-injecting and electron-injecting electrodes which are arranged adjacent one another, typically the organic layer or layers are sandwiched between the electrode layers. In order that light generated in the light emission layer can escape the EL device either the substrate-side (including the substrate) and/or the side facing away form the substrate is made transparent to the light to be emitted. To prevent ingress of oxygen and water the EL device is generally enclosed in an air and waterproof enclosure. Typically the enclosure comprises the substrate and a lid or cover foil glued to the substrate. Epoxy glue may be suitably used provided water getter material is provided to absorb moisture entering the device via the epoxy glue seal.

The electroluminescent device may be used for lighting applications such as indicator lighting, lit billboards or back-lights. The electroluminescent device may also be used as a display such as a segmented display, a pixelated passive matrix or active matrix display. The display may be monochrome, multi-color or even full-color. The combinations of carbazole compounds and light-emissive compound are of particular use in multi-color and full-color displays as the same carbazole compound can be used to in each pixel, only the light-emissive triplet compound needs changing if a different color is desired.

The electroluminescent display devices may be used for hand-held devices such as mobile phones, personal digital assistants and palmtops, notebook computers desktop displays and television applications. Projection systems may also comprise an electroluminescent device.

These and other aspects of the invention will be apparent from and elucidated with reference to the drawings and the embodiments described hereinafter.

In the drawings:

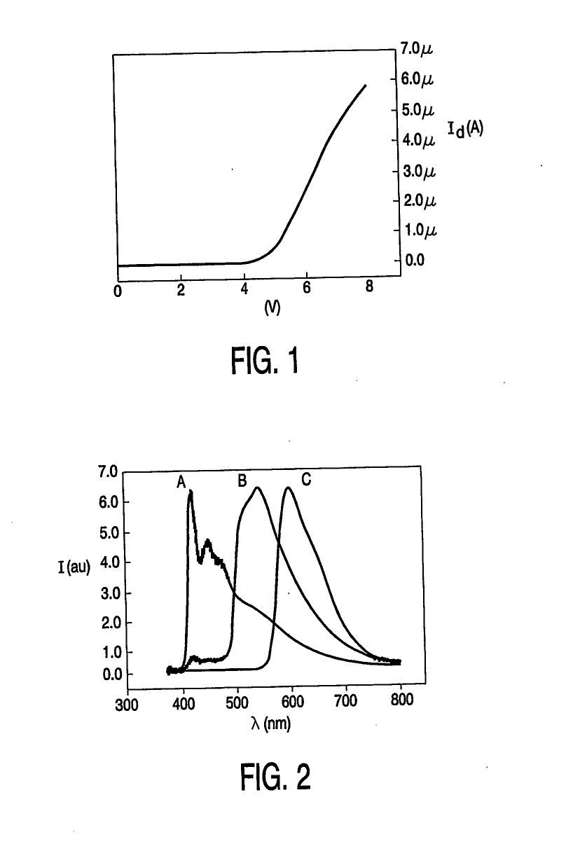

FIG. 1 shows a graph of the photodiode current I_{d} (in A) versus the voltage V (in V) impressed on an electroluminescent device comprising a polymer comprising a carbazole multimer unit in accordance with the invention;

FIG. 2 shows electroluminescence spectra labeled A, B and C of electroluminescent devices comprising carbazole compounds in accordance with the invention; and

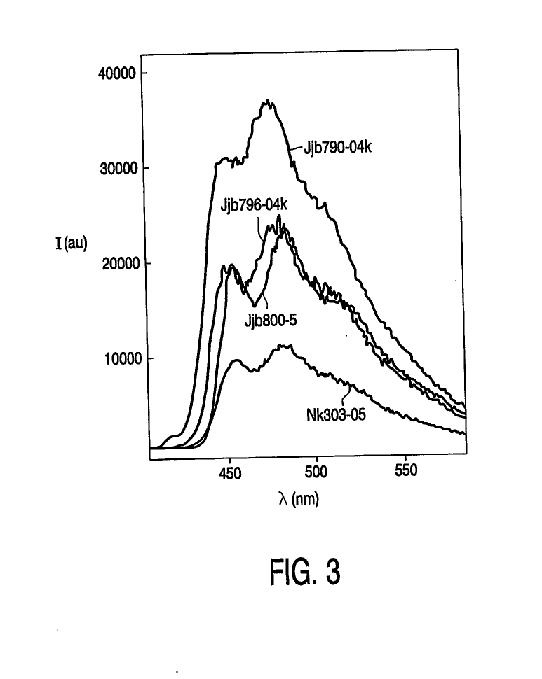

FIG. 3 shows phosphorescence emission spectra of a series of carbazole compounds in accordance with the invention.

### SYNTHETIC EXAMPLE 1

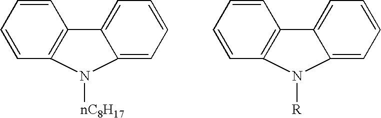

### 9-octylcarbazole

To a stirred solution of 20.0 g (0.12 mol) carbazole and 0.8 g benzyltriethylammoniumchloride in 100 ml toluene 70 g 50 w % NaOH (aq) is added, after which 27.7 g (0.14 mol) octylbromide is added dropwise. After complete addition the reaction mixture is heated to reflux during 16 hours. The toluene fraction was separated, washed with water, dried over MgSO₄, filtered and concentrated. Pure product was obtained after column chromatography (SiO₂, hexane/triethylamine, 98/2, v/v) as a sticky solid, 31.5 g (94%).

¹H NMR (CDCl₃): δ8.15 (dd, J=1.5 Hz, J=8 Hz, 2H), 7.50 (dt, J=1.5 Hz, J=8 Hz, 2H), 7.45 (d, J=8 Hz, 2H), 7.26 (dt, J=1.5 Hz, J=8 Hz, 2H), 4.35 (t, J=8 Hz, 2H), 1.95-1.85 (m, 2H), 1.50-1.20 (m, 10H), 0.92 (t, J=6.5 Hz, 3H).

¹³C NMR (CDCl₃): δ140, 126, 123, 120, 119, 109, 43, 32, 29, 29, 29, 27, 23, 14.

### 9-(3,7-dimethyloctyl)carbazole

### Synthesis analogous to 9-octylcarbazole

¹H NMR (CDCl₃): δ8.23 (dd, J=1.5 Hz, J=8 Hz, 2H), 7.58 (dt, J=1.5 Hz, J=8 Hz, 2H), 7.51 (d, J=8 Hz, 2H), 7.35 (dt, J=1.5 Hz, J=8 Hz, 2H), 4.47-4.35 (m, 2H), 2.00-1.90 (m, 1H), 1.85-1.20 (m, 9H), 1.15 (d, J=6.5 Hz, 3H), 1.00 (d, J=6.5 Hz, 6H).

¹³C NMR (CDCl₃): δ140, 126, 123, 120, 119, 109, 41, 39, 37, 36, 31, 28, 25, 23, 23, 20.

### SYNTHETIC EXAMPLE 2

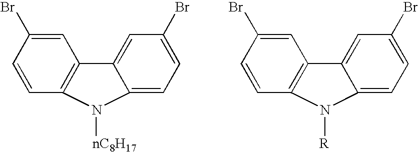

### 3,6-dibromo-9-octylcarbazole (nk202)

A stirred solution of 10.0 g (35.8 mmol) 9-octylcarbazole in 200 ml tetrahydrofuran is cooled to 0° C. 12.4 g (69.8 mmol) N-bromosuccinimide is added in small portions. The mixture is allowed to warm to room temperature overnight. The TBF is evaporated and the product is purified by an extraction with diethyl ether and water. The organic layer is dried (MgSO₄), filtered, concentrated and further purified by column chromatography (SiO₂, hexane/dichloromethane, 95/5, v/v) and crystallization (hexane/dichloromethane), respectively, yielding 12.2 g (78%) white crystals.

¹H NMR (CDCl₃): δ8.13 (d, J=1.5 Hz, 2H), 7.57 (dd, J=1.5 Hz, J=8 Hz, 2H), 7.25 (d, J=8 Hz, 2H), 4.35 (t, J=8 Hz, 2H), 1.95-1.85 (m, 2H), 1.50-1.20 (m, 10H), 0.92 (t, J=6.5 Hz, 3H).

¹³C NMR (CDCl₃): δ139, 129, 123, 123, 112, 110, 43, 32, 29, 29, 29, 27, 23, 14.

### 3,6-dibromo-9-(3,7-dimethyloctyl)carbazole

### Synthesis analogous to 3,6-dibromo-9-octylcarbazole

¹H NMR (CDCl₃): δ8.13 (d, J=1.5 Hz, 2H), 7.57 (dd, J=1.5 Hz, J=8 Hz, 2H), 7.25 (d, J=8 Hz, 2H), 4.30-4.15 (m, 2H), 1.88-1.75 (m, 1H), 1.65-1.10 (m, 9H), 1.05 (d, J=6.5 Hz, 3H), 0.90 (d, J=6.5 Hz, 6H).

¹³C NMR (CDCl₃): δ139, 129, 123, 123, 112, 110, 41, 39, 37, 35, 31, 28, 25, 23, 23, 20.

### SYNTHETIC EXAMPLE 3

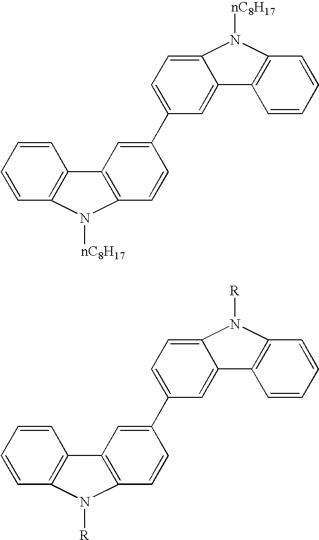

### bis[9-octylcarbazol-3-yl] (jjib790-04k)

To a stirred solution of 3.23 g (11.6 mmol) 9-octylcarbazole in 50 ml chloroform under argon atmosphere is added at once 3.75 g (23.2 mmol) iron(III)chloride. After stirring at room temperature during 16 hours 50 ml water are added. The organic layer was separated, dried over MgSO₄, filtered and concentrated. The mixture was purified by column chromatography (SiO₂, hexane/dichloromethane/triethylamine, 80/20/1, v/v/v) and crystallization (hexane/dichloromethane), respectively. 2.59 gram (81%) of product was obtained as white crystals.

¹H NMR (CDCl₃): 8.46 (d, J=1.5 Hz, 2H), 8.24 (d, J=8 Hz, 2H), 7.88 (dd, J=1.5 Hz, J=8 Hz, 2H), 7.57-7.46 (m, 6H), 7.30 (dt, J=1.5 Hz, J=8 Hz, 2H), 4.38 (t, J=8 Hz, 4H), 2.00-1.90 (m, 4H), 1.52-1.22 (m, 20H), 0.92 (t, J=6.5 Hz, 6H).

¹³C NMR (CDCl₃): δ141, 140, 133, 126, 126, 123, 123, 120, 119, 119, 109, 109, 43, 32, 29, 29, 29, 27, 23, 14.

### bis[9-(3,7-dimethyloctyl)carbazol-3-yl]

### Synthesis analogous to bis[9-octylcarbazol-3-yl] (jib790-04k)

¹HNMR (CDCl₃): δ8.50 (s, 2H), 8.28 (d, J=8 Hz, 2H), 7.91 (dd, J=1.5 Hz, J=8 Hz, 2H), 7.59-7.55 (d+t, 4H), 7.49 (d, J=8 Hz, 2H), 7.34 (t, J=8 Hz, 2H), 4.48-4.35 (m, 4H), 2.05-1.90 (m, 2H), 1.82-1.20 (m, 18H), 1.14 (d, J=6.5 Hz, 6H), 0.96 (d, J=6.5 Hz, 12H).

¹³C NMR (CDCl₃): δ141, 139, 133, 126, 126, 123, 123, 121, 119, 119, 109, 109, 41, 39, 37, 36, 31, 28, 25, 23, 23, 20.

### SYNTHETIC EXAMPLE 4

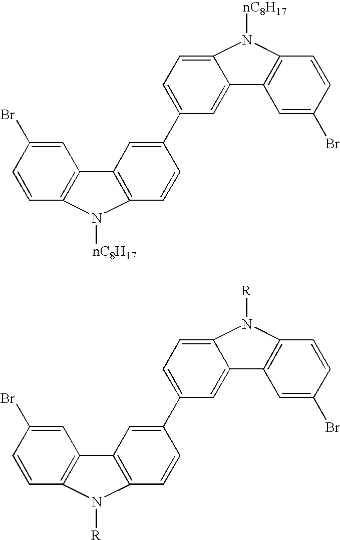

### bis[6-bromo-9-octylcarbazol-3-yl] (nk243)

A stirred solution of 4.42 g (7.94 mmol) bis(9-octylcarbazol-3-yl) in 200 ml tetrahydrofuran is cooled to 0° C. 2.82 g (15.8 mmol) N-bromosuccinimide is added in small portions. The mixture is allowed to warm to room temperature overnight. The TPF is evaporated and the product is purified by extraction with diethyl ether and water. The organic layer is dried (MgSO₄), filtered, concentrated and further purified by column chromatography (SiO₂, hexane/dichloromethane, 95/5, v/v) and crystallization (hexane/dichloromethane), respectively, yielding 3.6 g (64%) of white powder.

¹HNMR (CDCl₃): δ8.36 (d, J=1.5 Hz, 2H), 8.32 (d, J=1.5 Hz, 2H), 7.86 (dd, J=1.5 Hz, J=8 Hz, 2H), 7.59 (dd, J=1.5 Hz, J=8 Hz, 2H), 7.51 (d, J=8 Hz, 2H), 7.32 (d, J=8 Hz, 2H), 4.35-4.25 (m, 4H), 1.97-1.83 (m, 4H), 1.52-1.22 (m, 20H), 0.92 (t, J=6.5 Hz, 6H).

¹³C NMR (CDCl₃): δ140, 139, 133, 128, 126, 125, 123, 122, 119, 112, 110, 109, 43, 32, 29, 29, 29, 27, 23, 14.

### bis[6-bromo-9-(3,7-dimethyloctyl)carbazol-3-yl]

A stirred solution of 5.08 g (8.30 mmol) bis[9-(3,7-dimethyloctyl)carbazol-3-yl] in 200 ml tetrahydrofuran is cooled to 0° C. 2.92 g (16.5 mmol) N-bromosuccinimide is added is small portions. The mixture is allowed to warm to room temperature overnight. The THF is evaporated and the product is purified by extraction with diethyl ether and water. The organic layer is dried (MgSO₄), filtered, concentrated and further purified by column chromatography (SiO₂, hexane/dichloromethane, 95/5, v/v), followed by crystallization (hexane/dichloromethane),yielding 5.1 g (58%) of white powder.

¹H NMR (CDCl₃): δ8.35 (d, J=1.5 Hz, 2H), 8.32 (d, J=1.5 Hz, 2H), 7.86 (dd, J=1.5 Hz, J=8 Hz, 2H), 7.59 (dd, J=1.5 Hz, J=8 Hz, 2H), 7.50 (d, J=8 Hz, 2H), 7.31 (d, J=8 Hz, 2H), 4.42-4.25 (m, 4H), 1.98-1.85 (m, 2H), 1.75-1.15 (m, 18H), 1.09 (d, J=6.5 Hz, 6H), 0.91 (d, J=6.5 Hz, 12H).

¹³C NMR (CDCl₃): δ140, 139, 133, 128, 126, 125, 123, 122, 119, 111, 110, 109, 41, 39, 37, 36, 31, 28, 25, 23, 23, 20.

### SYNTHETIC EXAMPLE 5

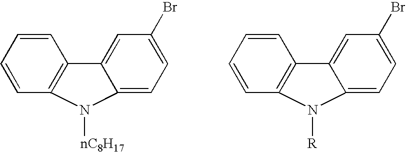

### 3-bromo-9-octy[carbazole

A stirred solution of 10 g (35.8 mmol) of 9-octylcarbazol in 400 ml tetrahydrofuran is cooled to 0° C. 3.83 g (21.5 mmol) N-bromosuccinimide is added in small portions. The mixture is allowed to warm to room temperature overnight The THF is evaporated and the product is purified by an extraction with diethyl ether and saturated aqueous solution of Na₂CO₃. The organic layer is dried (MgSO₄), filtered and concentrated, yielding 9.91 g of a pale yellow oil (mixture of starting compound and mono brominated product).

¹H NMR (CDCl₃): δ8.26 (d, J=1.5 Hz, 1H), 8.12 (d, J=1.5 Hz, J=8 Hz, 1H), 7.59 (dd, J=1.5 Hz, J=8 Hz, 1H), 7.54 (dt, J=1.5 Hz, J=8 Hz, 1H), 7.46 (d, J=8 Hz, 1H), 7.36-7.26 (m, 2H), 4.28 (t, J=8 Hz, 2H), 1.95-1.82 (m, 2H), 1.45-1.20 (m, 10H), 0.95 (t, J=6.5 Hz, 3H).

¹³C NMR (CDCl₃): δ140, 139, 128, 126, 124, 123, 122, 120, 119, 111, 110, 109, 43, 32, 29, 29, 29, 27, 23, 14.

### SYNTHETIC EXAMPLE 6

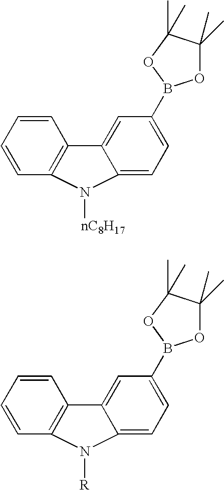

### 3-(4,4,5,5-tetramethyl-1,3,2-dioxaborolyl)-9-octylcarbazole

A solution of 1.75 g (4.9 mmol) of 3-bromo-9-octylcarbazole in 50 ml tetrahydrofuran is cooled to −70° C. 2.5 ml (6.2 mmol) 2.5 M n-butyllithium is added dropwise. After 1 hour 1.3 ml (6.3 mmol) of 2-isopropoxy-4,4,5,5-tetramethyl-1,3,2-dioxaborolane is added dropwise. The mixture is allowed to warm to room temperature overnight. The THF is evaporated and the product is purified by an extraction with diethyl ether and water. The organic layer is dried (MgSO₄), filtered, concentrated and further purified by column chromatography (SiO₂, hexane/dichloromethane/triethylamine, 60/40/1, v/v/v), yielding 1.6 g (81%) product as a colorless oil.

¹H NMR (CDCl₃): δ8.66 (d, J=1.5 Hz, 1H), 8.20 (dd, J=1.5 Hz, J=8 Hz, 1H), 7.99 (dd, J=1.5 Hz, J=8 Hz, 1H), 7.56-7.44 (m, 3H), 7.30 (dt, J=1.5 Hz, J=8 Hz, 2H), 4.38 (t, J=8 Hz, 2H), 2.00-1.85 (m, 2H), 1.55-1.20 (m, 10H), 1.46 (s, 12H), 0.95 (t, J=6.5 Hz, 3H).

¹³C NMR (CDCl₃): δ142, 140, 132, 128, 125, 123, 122, 120, 119, 109, 108, 83, 43, 32, 29, 29, 29, 27, 25, 23, 14.

### SYNTHETIC EXAMPLE 7

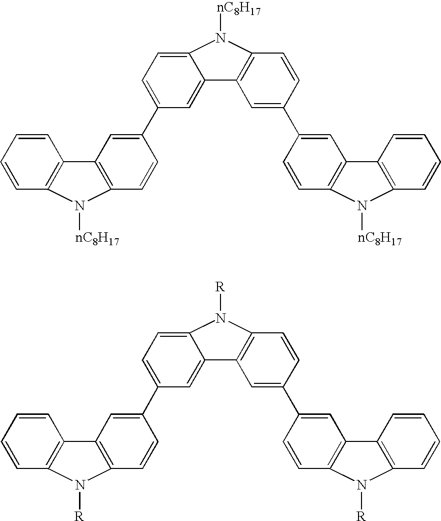

### [3,3′:6′,3′]tris(9-octylcarbazole) (nk303-05 or nk29308)

A flask containing a mixture of 0.93 g (2.1 mmol) 3,6-dibromo-9-octylcarbazole, 1.90 g (4.7 mmol) 3-(4,4,5,5-tetramethyl-1,3,2-dioxaborolyl)-9-octylcarbazole, 20 ml (20 mmol) 1 M K₂CO₃ (aq) in 20 ml toluene was evacuated and charged with argon for three times, after which 2 mol % Pd(PPh₃)₄ was added. Evacuation and filling with argon was repeated once and the mixture was stirred for 16 hours at reflux temperature. The mixture was allowed to cool to room temperature and water was added. The organic layer was separated, dried (MgSO₄), filtered and concentrated. After column chromatography (SiO₂, hexane/dichloromethane, 80/20, v/v) and crystallization (hexane/dichloromethane) respectively, 1.38 g (78%) of product was obtained.

¹H NMR (CDCl₃): δ8.58 (d, J=1.5 Hz, 2H), 8.52 (d, J=1.5 Hz, 2H), 8.27 (d, J=8 Hz, 2H), 7.59 (dd, J=1.5 Hz, J=8 Hz, 2H), 7.92 (dd, J=1.5 Hz, J=8 Hz, 2H), 7.57 (d, J=8 Hz, 2H), 7.56 (d, J=8 Hz, 2H), 7.55 (dt, J=1.5 Hz, J=8 Hz, 2H), 7.49 (d, J=8 Hz, 2H), 7.32 (dt, J=1.5 Hz, J=8 Hz, 2H), 4.45-4.35 (m, 6H), 2.05-1.90 (m, 6H), 1.55-1.22 (m, 30H), 0.98-0.90 (m, 9H).

¹³C NMR (CDCl₃): δ141, 140, 139, 133, 133, 125, 125, 125, 124, 123, 123, 120, 119, 119, 119, 109, 109, 109, 43, 43, 32, 32, 30, 30, 29, 29, 29, 29, 27, 27, 23, 14.

### SYNTHETIC EXAMPLE 8

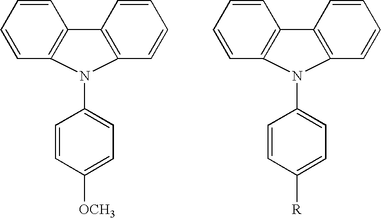

### 9-(4-methoxyphenyl)carbazole

A flask containing a mixture of 20.1 g (0.12 mol) carbazole, 29.2 g (0.16 mol) 4-bromoanisole, 50 g (0.36 mol) K₂CO₃ in 200 ml toluene was evacuated and charged with argon for three times, after which 2 mol % Pd(OAc)2 and 0.4 g tris(tert.butyl)phosphine were added. Evacuation and filling with argon was repeated once and the mixture was stirred for one week at reflux temperature (after three days 7.5 g anisole and some fresh Pd(OAc)₂ and PtBu₃ were added). The mixture was allowed to cool to room temperature and water was added. The organic layer was separated, dried (MgSO₄), filtered and concentrated. After column chromatography (SiO₂, hexane/dichloromethane/triethylamine, 80/20/1, v/v/v) 23.92 g (73%) of product was obtained.

¹H NMR (CDCl₃): δ8.23 (d, J=8 Hz, 2H), 7.53 (d, J=8 Hz, 2H), 7.49 (dt, J=1.5 Hz, J=8 Hz, 2H), 7.42 (d, J=8 Hz, 2H), 7.36 (dt, J=1.5 Hz, J=8 Hz, 2H), 7.18 (d, J=8 Hz, 2H), 3.98 (s, 3H).

¹³C NMR (CDCl₃): δ159, 141, 130, 129, 126, 123, 120, 120, 115, 110, 56.

### SYNTHETIC EXAMPLE 9

### 3-bromo-9-(4-methoxyphenyl)carbazole

### Synthesis Analogous to Synthetic Example 5

¹H NMR (CDCl₃): δ8.29 (d, J=1.5 Hz, 1H), 8.13 (dd, J=1.5 Hz, J=8 Hz, 1H), 7.51 (d, J=1.5 Hz, J=8 Hz, 1H), 7.46 (dt, J=1.5 Hz, J=8 Hz, 1H), 7.45 (d, J=8 Hz, 2H), 7.35 (d, J=8 Hz, 1H), 7.33 (dt, J=1.5 Hz, J=8 Hz, 1H), 7.23 (d, J=8 Hz, 1H), 7.15 (d, J=8 Hz, 2H), 3.96 (s, 3H).

¹³C NMR (CDCl₃): δ159, 141, 140, 129, 128, 128, 127, 125, 123, 122, 120, 120, 115, 112, 111, 110, 56.

### SYNTHETIC EXAMPLE 10

### 3-(4,4,5,5-tetramethyl-1,3,2-dioxaborolyl)-9-(4-methoxyphenyl)carbazole

### Synthesis Analogous to Synthetic Example 6

¹H NMR (CDCl₃): δ8.69 (d, J=1.5 Hz, 1H), 8.23 (dd, J=1.5 Hz, J=8 Hz, 1H), 7.91 (d, J=1.5 Hz, J=8 Hz, 1H), 7.44 (dt, J=1.5 Hz, J=8 Hz, 1H), 7.39 (d, J=8 Hz, 2H), 7.37 (d, J=8 Hz, 1H), 7.36 (d, J=8 Hz, 1H), 7.34 (dt, J=1.5 Hz, J=8 Hz, 1H), 7.16 (d, J=8 Hz, 2H), 3.96 (s, 3H), 1.46 (s, 12H).

¹³C NMR (CDCl₃): δ159, 143, 142, 132, 130, 129, 128, 126, 123, 123, 121, 120, 120, 115, 110, 109, 94, 84, 56, 25.

### SYNTHETIC EXAMPLE 10a

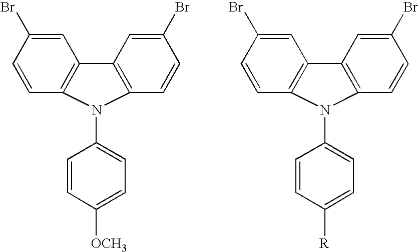

### 3,6-dibromo9-(4-methoxyphenyl)carbazole (nk26803)

### Synthesis Analogous to Synthetic Example 2

¹H NMR (CDCl₃): δ8.21 (d, J=1.5 Hz, 2H), 7.52 (dd, J=1.5 Hz, J=8 Hz, 2H), 7.39 (d, J=8 Hz, 2H), 7.20 (d, J=8 Hz, 2H), 7.17 (d, J=8 Hz, 2H), 3.95 (s, 3H).

¹³C NMR (CDCl₃): δ159, 140, 129, 129, 128, 124, 123, 115, 113, 111, 56.

### SYNTHETIC EXAMPLE 11

### bis[9-(4-methoxyphenyl)carbazol-3-yl] (jjb796-04k)

To a stirred solution of 2.49 g (9.1 mmol) 9-(4-methoxyphenyl)-carbazole in 70 ml chloroform under argon atmosphere is added at once 3.0 g (18.5 mmol) iron(III)chloride. After stirring at room temperature during 40 hours 75 ml water are added. The organic layer was separated, dried over MgSO₄, filtered and concentrated. The mixture was purified by column chromatography (SiO₂, hexane/dichloromethane, 60/40, v/v) and crystallization (hexane/dichloromethane), respectively. 1.29 gram (52%) of white crystals was obtained.

¹H NMR (CDCl₃): δ8.49 (d, J=1.5 Hz, 2H), 8.28 (d, J=8 Hz, 2H), 7.81 (dd, J=1.5 Hz, J=8 Hz, 2H), 7.54 (d, J=8 Hz, 4H), 7.47 (d, J=8 Hz, 2H, 7.46 (dt, J=1.5 Hz, J=8 Hz, 2H), 7.40 (d, J=8 Hz, 2H), 7.35 (dt, J=1.5 Hz, J=8 Hz, 2H), 7.18 (d, J=8 Hz, 4H), 3.97 (s, 6H).

¹³C NMR (CDCl₃): δ159, 142, 141, 134, 130, 129, 126, 126, 124, 123, 120, 120, 119, 115, 110, 110, 56.

### SYNTHETIC EXAMPLE 12

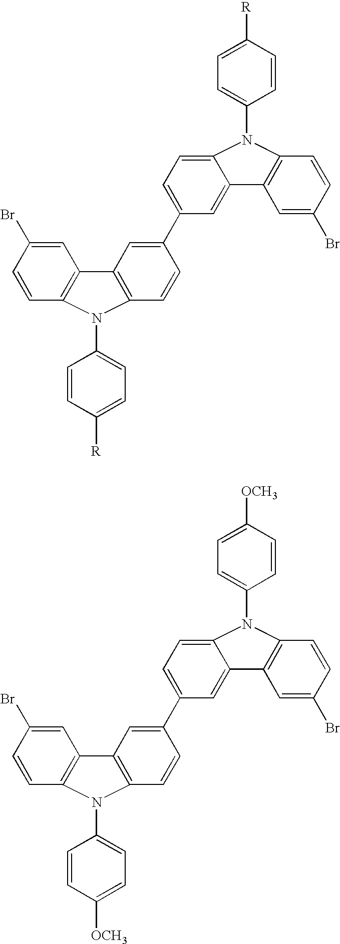

### bis[6-bromo-9-(4-methoxyphenyl)carbazol3-yl] (nk27003)

### Synthesis Analogous to Synthetic Example 4

¹H NMR (CDCl₃): δ8.41 (d, J=1.5 Hz, 2H), 8.38 (d, J=1.5 Hz, 2H), 7.79 (dd, J=1.5 Hz, J=8 Hz, 2H), 7.53 (dd, J=1.5 Hz, J=8 Hz, 2H), 7.48 (d, J=8 Hz, 4H), 7.44 (d, J=8 Hz, 2H), 7.26 (d, J=8 Hz, 2H), 7.17 (d, J=8 Hz, 4H), 3.97 (s, 6H).

¹³C NMR (CDCl₃): δ159, 141, 140, 134, 130, 129, 128, 126, 125, 123, 123, 119, 115, 113, 111, 110, 56.

### SYNTHETIC EXAMPLE 13

### [3,3′:6′,3″]tris(9-(4methoxyphenyl)carbazole) (jjb800-05)

### Synthesis Analogous to Synthetic Example 7

¹H NMR (CDCl₃): δ8.65 (d, J=1.5 Hz, 2H), 8.58 (d, J=1.5 Hz, 2H), 8.37 (d, J=8 Hz, 2H), 7.87 (dd, J=1.5 Hz, J=8 Hz, 4H), 7.62-7.37 (m, 16H), 7.26 (d, J=8 Hz, 2H), 7.17 (d, J=8 Hz, 4H), 4.00 (s, 3H), 3.98 (s, 6H).

¹³C NMR (CDCl₃): δ159, 142, 141, 140, 134, 134, 130, 130, 128, 128, 126, 126, 126, 124, 124, 123, 120, 120, 119, 119, 115, 115, 110, 110, 110, 56.

### SYNTHETIC EXAMPLE 14

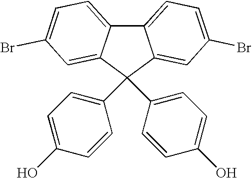

### 2,7-dibromo-9,9-bis(4-hydroxyphenyl)fluorene

A mixture of 9.2 g (0.027 mol) 2,7-dibromofluorenone, 17.0 g (0.18 mol) phenol and 7.8 g (0.08 mol) methanesulfonic acid in 40 g tetrachloromethane was stirred at 80° C. during 40 hours. The mixture was allowed to cool to room temperature, after which the product was filtered and washed with dichloromethane. Yield 11.0 g (80%) of pale red powder.

¹H NMR (CDCl₃): δ7.86 (d, J=8 Hz, 2H), 7.60 (d, J=1.5 Hz, 2H), 7.60 (dd, J=1.5 Hz, J=8 Hz, 2H), 7.05 (d, J=8 Hz, 4H), 6.80 (d, J=8 Hz, 4H).

¹³C NMR (CDCl₃): δ157, 155, 138, 135, 131, 129, 129, 122, 122, 115

### SYNTHETIC EXAMPLE 15

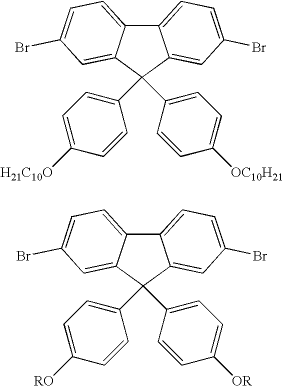

### 2,7-dibromo-9,9-bis[4-(3,7-dimethyloctyloxy)phenyl]fluorene

A mixture of 11.0 g (21.7 mmol) 2,7-dibromo-9,9-bis(4-hydroxyphenyl)fluorene, 10.6 (48.1 mmol) 3,7-ditnethyloctylbromide, 5.4 (39 mmol) K₂CO₃ in 200 ml methylisobutylketone was stirred at reflux during 40 hours. The solvent was evaporated and the product was isolated by dichloromethane/water extraction. The organic layer was dried (MgSO₄), filtered and concentrated. Purification by column chromatography (SiO₂, hexane/dichloromethane, 85/15, v/v) yielded 10.2 g (60%) of product as off-white solid.

¹H NMR (CDCl₃): δ7.56 (d, J=8 Hz, 2H), 7.47 (d, J=1.5 Hz, 2H), 7.45 (dd, J=1.5 Hz, J=8 Hz, 2H), 7.05 (d, J=8 Hz, 4H), 6.76 (d, J=8 Hz, 4H), 4.00-3.88 (m, 4H), 1.85-1.75 (m, 2H), 1.70-1.10 (m, 18H), 0.95 (d, J=8 Hz, 6H), 0.90 (d, J=8 Hz, 12H).

¹³C NMR (CDCl₃): δ158, 154, 138, 136, 131, 129, 129, 122, 121, 114, 66, 39, 37, 36, 30, 28, 27, 25, 23, 23, 20.

### SYNTHETIC EXAMPLE 16

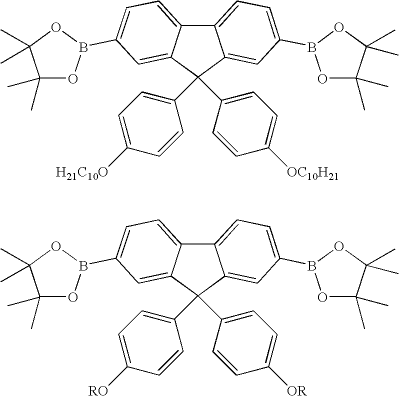

### 2,7-bis(4,4,5,5-tetramethyl-1,3,dioxaborolyo)-9,9-bis[4-(3,7-dimethyloctyloxy)phenyl]fluorene

A solution of 24.38 g ( 30.9 mmol) 2,7-dibromo-9,9-bis[4-(3,7-dimethyloctyloxy)phenyl]fluorene in 300 ml tetrahydrofuran was stirred at −70° C. under argon atmosphere. 33 ml (82.5 mmol) 2.5 M n-butyllithium was added dropwise. After 1.5 hour 16.5 ml (80.5 mmol) 2-isopropoxy-4,4,5,5-tetramethyl-1,3,2-dioxaborolane was added dropwise. The mixture was allowed to warm to room temperature overnight. The THF was evaporated and the product was isolated by diethylether/water extraction. The organic layer was dried (MgSO₄), filtered and concentrated. The product was purified by crystallization from hexane/diethyl ether, yielding 19.1 g (70%) product as white crystals.

¹H NMR (CDCl₃): δ7.82-7.74 (m, 6H), 7.12 (d, J=8 Hz, 4H), 6.73 (d, J=8 Hz, 4H), 3.95-3.84 (m, 4H), 1.85-1.70 (m, 2H), 1.68-1.10 (m, 18H), 1.30 (s, 12H), 0.95 (d, J=8 Hz, 6H), 0.92 (d, J=8 Hz, 12H).

¹³C NMR (CDCl₃): δ158, 152, 143, 138, 134, 132, 129, 120, 114, 84, 66, 39, 37, 36, 30, 28, 25, 25, 23, 23, 20.

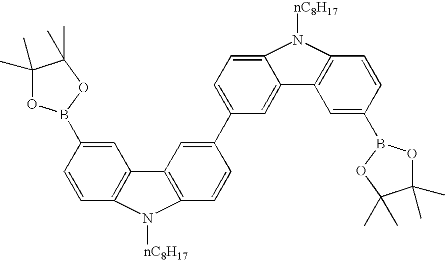

### bis[3-{4,4,5,5-tetramethyI-1,3,2-dioxaborolyl}-9-octylcarbazol-6-yl]

Synthesis analogous to 2,7-bis(4,4,5,5-tetramethyl- 1,3,2-dioxaborolyl)-9,9-bis[4-(3,7-dimethyloctyloxy)phenyl]fluorene

¹H NMR (CDCl₃): δ8.79 (s, 2H), 8.58 (d, J=1.5 Hz, 2H), 8.01 (d, J=8 Hz, 2H), 7.92 (dd, J=1.5 Hz, J=8 Hz, 2H), 7.58 (d, J=8 Hz, 2H), 7.49 (d, J=8 Hz, 2H), 4.41 (t, J=8 Hz, 4H), 2.01-1.90 (m, 4H), 1.50-1.20 (m, 20H), 1.42 (s, 24H), 0.95 (d, J=6.5 Hz, 6H).

¹³C NMR (CDCl₃): δ143, 140, 133, 132, 128, 125, 124, 123, 119, 109, 108, 83, 43, 32, 29, 9, 29, 27, 25, 23, 14.

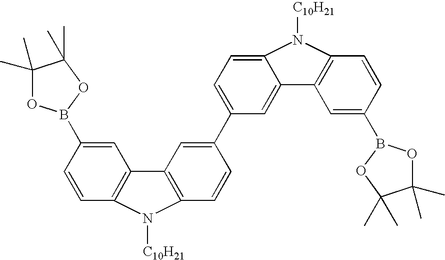

### bis[3-{4,4,5,5-tetramethyl-1,3,2-dioxaborolyl}-9-(3,7-dimethyloctyl)carbazol-6-yl]

Synthesis analogous to 2,7-bis(4,4,5,5-tetramethyl- 1,3,2-dioxaborolyl)-9,9-bis[4-(3,7-dimethyloctyloxy)phenyl]fluorene

¹H NMR (CDCl₃): δ8.77 (s, 2H), 8.55 (d, J=1.5 Hz, 2H), 8.00 (d, J=1.5 Hz, J=8 Hz, 2H), 7.90 (dd, J=1.5 Hz, J=8 Hz, 2H), 7.54 (d, J=8 Hz, 2H), 7.46 (d, J=8 Hz, 2H), 4.48-4.35 (m, 4H), 2.03-1.90 (m, 2H), 1.80-1.05 (m, 18H), 1.42 (s, 24H), 1.10 (d, J=6.5 Hz, 6H), 0.92 (d, J=6.5 Hz, 12H).

¹³C NMR (CDCl₃): δ143, 140, 133, 132, 128, 125, 124, 123, 119, 109, 108, 84, 41, 39, 37, 36, 31, 28, 25, 25, 23, 23, 20.

### SYNTHETIC EXAMPLE 17

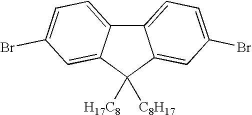

### 2,7-dibromo-9,9-dioctylfluorene

To a mixture of 40.2 g (124 mmol) 2,7-dibromofluorene and 1.80 g tetrabutylammonium hydroxide in 80 ml DMSO was added dropwise 40 ml 50 w % NaOH (aq), followed by 51.4 g (266 mmol) octylbromide. The mixture was heated at 80° C. during 48 hours. The DMSO was evaporated, and the product was isolated by diethyl ether/1 M HCl (aq) extraction. The organic layer was dried (MgSO₄), filtered and concentrated. The product was purified by crystallization from ethanol/dichloromethane, resulting in 51.4 g (76%) white crystals.

¹H-NMR (CDCl₃): δ7.51 (d, J=8 Hz, 2H), 7.45 (d, J=8 Hz, 2H), 7.44 (s, 2H), 1.93-1.90 (m, 4H), 1.26-1.05 (m, 20H), 0.83 (t, J=6.5 Hz, 6H), 0.60-0.56 (m, 4H).

¹³C-NMR (CDCl₃): δ153, 139, 130, 126, 121, 121, 56, 40, 32, 30, 29, 24, 23, 14.

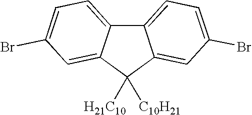

### 2,7-dibromo-9,9-bis(3,7-dimethyloctyl)fluorene

Synthesis analogous to 2,7-dibromo-9,9-dioctylfluorene

¹H-NMR (CDCl₃): δ7.52 (d, J=8 Hz, 2H), 7.44 (d, J=8 Hz, 2H), 7.43 (s, 2H), 1.96-1.89 (m, 4H), 1.55-0.86 (m, 16H), 0.82 (d, J=6.5 Hz, 12H), 0.69 (d, J=6.5 Hz, 6H), 0.56-0.53 (m, 2H), 0.44-0.42 (m, 2H).

¹³C-NMR(CDCl₃): δ152, 139, 130, 126, 121, 121, 56, 39, 38, 37, 33, 30, 28, 25, 23, 23, 20.

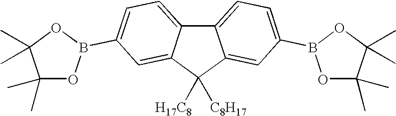

### 2,7-bis(4,4,5,5-tetramethyl-1,3,2-dioxaborolyl)-9,9-dioctylfluorene

Synthesis analogous to synthetic example 16 with 2,7-dibromo-9,9-dioctylfluorene as starting compound.

¹H-NMR (CDCl₃): δ7.86 (dd, J=1.5 Hz, J=8 Hz, 2H), 7.80 (d, J=1.5 Hz, 2H), 7.77 (d, J=8 Hz, 2H), 2.08-1.95 (m, 4H), 1.42 (s, 24H), 1.50-0.98 (m, 20H), 0.85 (t, J=6.5 Hz, 6H), 0.65-0.50 (m, 4H).

¹³C-NMR (CDCl₃): δ150, 144, 134, 129, 119, 84, 55, 40, 32, 30, 29, 29, 25, 23, 23, 14.

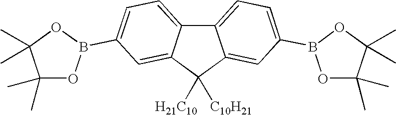

### 2,7-bis(4,4,5,5-tetramethyl-1,3,2-dioxaborolyl)-9,9-bis(3,7-dimethyloctyl)fluorene

Synthesis analogous to synthetic example 16 with 2,7-dibromo-9,9-bis(3,7-dimethyloctyl)fluorene as starting compound.

¹H-NMR (CDCl₃): δ7.86 (dd, J=1.5 Hz, J=8 Hz, 2H), 7.80 (d, J=1.5 Hz, 2H), 7.77 (d, J=8 Hz, 2H), 2.05-1.90 (m, 4H), 1.75-0.86 (m, 16H), 0.82 (d, J=6.5 H, 12H), 0.69 (d, J=6.5 Hz, 6H), 0.56-0.53 (m, 2H), 0.44-0.42 (m, 2H).

¹³C-NMR (CDCl₃): δ150, 144, 134, 129, 120, 119, 84, 55, 39, 37, 37, 33, 30, 28, 25, 23, 23, 20.

### SYNTHETIC EXAMPLE 18

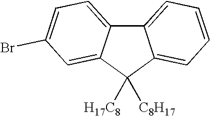

### 2-bromo-9,9-dioctylfluorene

### Synthesis Analogous to Synthetic Example 5

¹H-NMR (CDCl₃): δ7.71-7.68 (m, 1H), 7.59 (dd, J=1.5 Hz, J=8 Hz, 1H), 7.49 (d, J=1.5 Hz, 1H), 7.48 (dd, J=1.5 Hz, J=8 Hz, 1H), 7.38-7.34 (m, 3H), 2.05-1.90 (m, 4H), 1.30-1.00 (m, 20H), 0.85 (t, J=6.5 Hz, 6H), 0.70-0.55 (m, 4H).

¹³C-NMR (CDCl₃): δ152, 150, 140, 140, 130, 127, 127, 126, 123, 121, 121, 120, 55, 40, 32, 30, 29, 24, 23, 14.

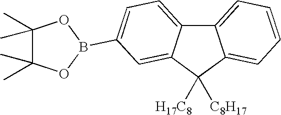

### 2-(4,4,5,5-tetramethyl-1,3,2-dioxaborolyl)-9,9-dioctylfluorene

### Synthesis Analogous to Synthetic Example 6

¹H-NMR (CDCl₃): δ7.84 (dd, J=1.5 Hz, J=8 Hz, 1H), 7.78-7.71 (m, 3H), 7.39-7.32 (m, 3H), 2.07-1.93 (m, 4H), 1.42 (s, 12H), 1.38-1.00 (m, 24H), 0.92 (t, J=6.5 H, 6H), 0.60-0.56 (m, 4H).

¹³C-NMR (CDCl₃): δ151, 150, 144, 141, 134, 129, 127, 127, 123, 120, 119, 84, 55, 40, 32, 32, 30, 29, 25, 24, 23, 14.

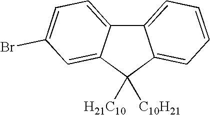

### 2-bromo-9,9-bis(3,7-dimethyloctyl)fluorene

### Synthesis Analogous to Synthetic Example 5

¹H-NMR (CDCl₃): δ7.71-7.66 (m, 1H), 7.59-7.55 (m, 1H), 7.49-7.44 (m, 2H), 7.37-7.31 (m, 3H), 2.05-1.90 (m, 4H), 1.90-1.00 (m, 18H), 0.92 (d, J=6.5 Hz, 12H), 0.70 (d, J=6.5 Hz, 6H), 0.68-0.38 (m, 2H).

¹³C-NMR (CDCl₃): δ152, 150, 140, 140, 130, 127, 127, 126, 123, 121, 121, 120, 56, 39, 38, 37, 33, 30, 28, 25, 23, 23, 20.

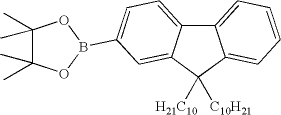

### 2-(4,4,5,5-tetramethyl-1,3,2-dioxaborolyl)-9,9-bis(3,7-dimethyloctyl)fluorene

### Synthesis Analogous to Synthetic Example 6

¹H-NMR (CDCl₃): δ7.81 (d, J=8 Hz, 1H), 7.74 (d, J=1.5 Hz, 1H), 7.73-7.70 (m, 1H), 7.69 (d, J=8 Hz, 1H), 7.35-7.28 (m, 3H), 2.07-1.93 (m, 4H), 1.90-0.80 (m, 16H), 1.38 (s, 12H), 0.80 (d, J=6.5 Hz, 12H), 0.63 (d, J=6.5 H, 6H), 0.60-0.35 (m, 4H).

¹³C-NMR (CDCl₃): δ151, 150, 144, 141, 134, 129, 127, 127, 123, 120, 119, 84, 55, 39, 37, 37, 33, 30, 28, 25, 23, 23, 20.

### SYNTHETIC EXAMPLE 19

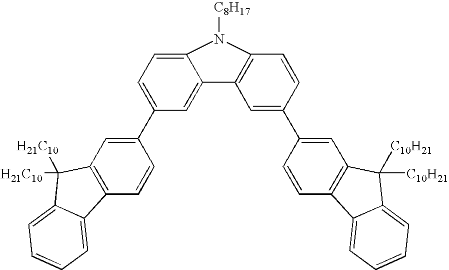

### 3,6-bis(9,9-[3,7-dimethyloctyl]fluoren-2-yl)-9-octylcarbazole (nk25320/25321)

A flask containing a mixture of 0.5 g (1.1 mmol) 3,6-dibromo-9-octylcarbazole, 1.4 g (2.4 mmol) 2-(4,4,5,5-tetramethyl-1,3,2-dioxaborolyl)-9,9-bis(3,7-dimethyloctyl)fluorene, 15 ml (1 M) K₂CO₃ (aq) and 15 ml toluene was evacuated and charged with argon for three times, after which 2 mol % Pd(PPh₃)₄ was added. Evacuation and filling with argon was repeated once and the mixture was stirred for 70 hours at reflux temperature. The mixture was allowed to cool to room temperature and the organic layer was separated, dried (MgSO₄), filtered and concentrated. After column chromatography (SiO₂, hexane/dichloromethane/triethylamine, 90/10/2, v/v/v) 0.65 g (49%) of product was obtained.

¹H NMR (CDCl₃): δ8.52 (d, J=1.5 Hz, 21H), 7.88-7.84 (m, 4H), 7.82-7.74 (m, 6H), 7.57 (d, J=8 Hz, 2H), 7.44-7.44 (m, 6H), 4.42 (t, J=8 Hz, 2H), 2.20-0.55 (m, 99H).

¹³C NMR (CDCl₃): δ152, 151, 141, 141, 140, 140, 133, 127, 127, 126, 125, 123, 123, 121, 120, 119, 119, 109, 55, 43, 39, 38, 37, 33, 32, 31, 30, 29, 29, 28, 27, 25, 23, 23, 23, 20, 14.

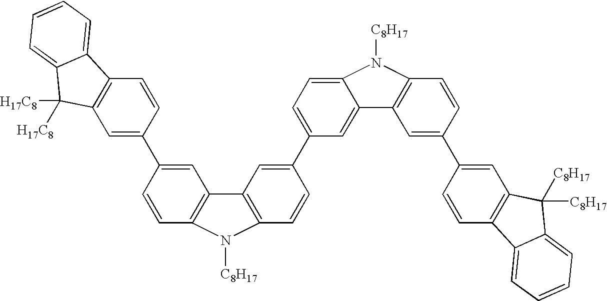

### bis[3-(9,9-dioctyl)fluoren-2-yl)-9-octylcarbazol-6-yl] (nk30006)

Synthesis analogous to 3,6-bis(9,9-[3,7-dimethyloctyl]fluoren-2-yl)-9-octylcarbazole with 2-(4,4,5,5-tetramethyl-1,3,2-dioxaborolyl)-9,9-dioctylfluorene and bis[6-bromo-9-octylcarbazol-3-yl] (nk243) as starting compounds.

¹H NMR (CDCl₃): δ8.56 (s, 2H), 8.52 (s, 2H), 7.93 (dd, J=1.5 Hz, J=8 Hz, 2H), 7.86 (dd, J=1.5 Hz, J=8 Hz, 2H), 7.83 (d, J=8 Hz, 2H), 7.80-7.73 (m, 6H), 7.58 (d, J=8 Hz, 2H), 7.55 (d, J=8 Hz, 2H), 7.42-7.32 (m, 6H), 4.43 (br s, 4H), 2.15-1.95 (m, 12H), 1.80-0.70 (m, 86H).

¹³C-NMR (CDCl₃): δ152, 150, 140, 140, 139, 130, 130, 129, 128, 127, 127, 126, 126, 125, 123, 123, 122, 121, 121, 120, 112, 112, 110, 109, 55, 43, 40, 32, 32, 30, 29, 29, 29, 27, 24, 23, 14.

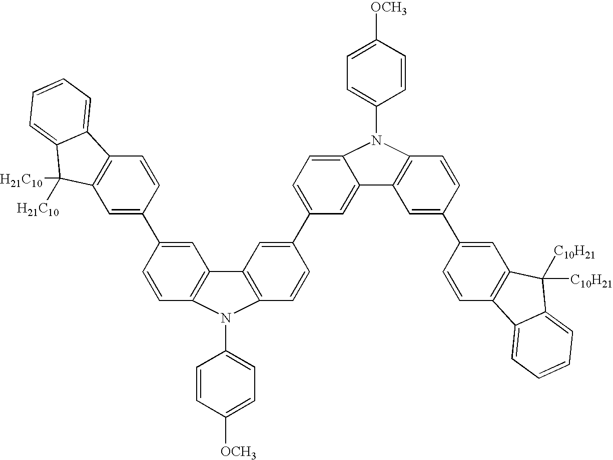

### bis(3-(9,9-[3,7-dimethyloctyl]fluoren-2-yl)-9-[4-methoxyphenyl]carbazol-6-yl} (nk27206)

¹H NMR (CDCl₃): δ8.63 (d, J=1.5 Hz, 2H), 8.60 (d, J=1.5 Hz, 2H), 7.94-7.75 (m, 12H), 7.63 (d, J=8 Hz, 4H), 7.56 (d, J=8 Hz, 2H), 7.52 (d, J=8 Hz, 2H), 7.46-7.35 (m, 6H), 7.22 (d, J=8 Hz, 4H), 4.00 (s, 6H), 2.20-2.00 (m, 8H), 1.80-0.95 (m, 32H), 0.93-0.50 (m, 44H).

¹³C NMR (CDCl₃): δ159, 151, 151, 141, 141, 141, 141, 140, 134, 134, 131, 128, 127, 127, 126, 126, 126, 124, 123, 122, 120, 120, 119, 119, 115, 110, 56, 55, 39, 38, 37, 33, 31, 28, 25, 23, 23, 20.

### SYNTHETIC EXAMPLE 20

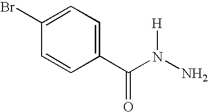

### 4-bromobenzoylhydrazine

A mixture of 31.2 g (145 mmol) methyl-4-bromobenzoate in 218 ml hydrazine.monohydrate was heated at 100° C. during 16 hours. Afterwards the mixture was allowed to cool and the product was filtered off and washed with water. Crystallization from ethanol yielded 19.4 g (62%) of white crystals.

¹H NMR (DMSO-d₆): δ10.5 (s, 1H), 7.79 (d, J=8 Hz, 2H), 7.70 (d, J=8 Hz, 2H).

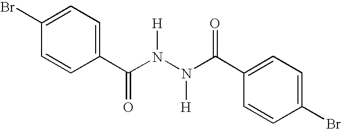

### 1,2-bis(4-bromobenzoyl)hydrazine

To 10.8 g (50 mmol) 4-bromobenzoylhydrazine in 80 ml (0.6 M) NaHCO₃ (aq) was added dropwise 11.0 g (50 mmol) 4-bromobenzoylchloride in 65 ml THF. The mixture was stirred during 16 hours, after which the product could be isolated by filtration. Yield 15.1 g (76%) white powder.

¹H NMR (DMSO-d₆): δ10.7 (s, 2H), 7.90 (d, J=8 Hz, 4H), 7.79 (d, J=8 Hz, 4H).

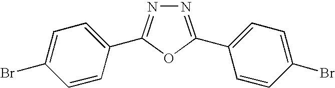

### 2,5-bis(4-bromophenyl)-1,3,4-oxadiazole

To 20.0 g (50 mmol) 1,2-bis(4-bromobenzoyl)hydrazine in 230 ml toluene was added cautiously 90 ml POCl₃, followed by stirring at reflux temperature during 16 hours. Afterwards the mixture was poured into a beaker containing ice and water. The organic layer was separated, dried (MgSO₄), filtered and concentrated. The product was purified by crystallization from ethanol, resulting in 15.4 g (81%) white crystals. ¹H NMR (CDCl₃): δ8.03 (d, J=8 Hz, 4H), 7.72 (d, J=8 Hz, 4H).

³C NMR (CDCl₃): δ164, 132, 128, 127, 123.

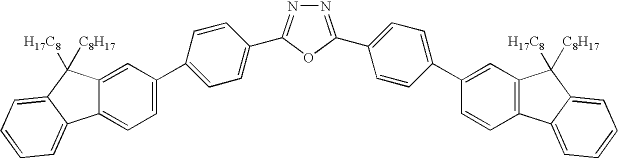

### 2,5-bis(4-[9,9-bisoctylfluoren-2-yl]phenyl)-1,3,4-oxadiazole

Synthesized using 2-(4,4,5,5-tetramethyl-1,3,2-dioxaborolyl)-9,9-dioctylfluorene and 2,5-bis(4-bromophenyl)-1,3,4-oxadiazole

¹H NMR (CDCl₃): δ8.30 (d, J=8 Hz, 4H), 7.89 (d, J=8 Hz, 4H), 7.85 (d, J=8 Hz, 2H), 7.79 (dd, J=1.5 Hz, J=8 Hz, 2H), 7.69 (dd, J=1.5 Hz, J=8 Hz, 2H), 7.66 (d, J=1.5 Hz, 2H), 7.43-7.35 (m, 6H), 2.10-2.00 (m, 8H), 1.30-1.05 (m, 40H), 0.85 (t, J=6.5 Hz, 12H), 0.80-0.65 (m, 8H).

¹³C NMR (CDCl₃): δ166, 152, 151, 145, 141, 140, 139, 128, 127, 127, 126, 123, 122, 121, 120, 120, 55, 40, 32, 30, 29, 24, 23, 14.

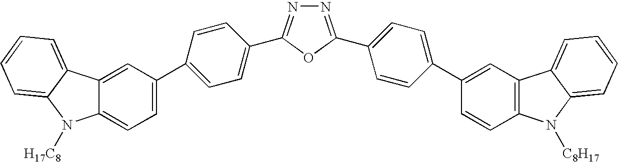

### 2,5-bis(4-[9-octylcarbazol-3-yl]phenyl)-1,3,4-oxadiazole

### Synthesized using 3-(4,4,5,5-tetramethyl-1,3,2-dioxaborolyl)-9-octylcarbazol and 2,5-bis(4-bromophenyl)-1,3,4-oxadiazole

¹H NMR (CDCl₃): δ8.42 (d, J=1.5 Hz, 2H), 8.26 (d, J=8 Hz, 4H), 8.22 (d, J=8 Hz, 2H), 7.90 (d, J=8 Hz, 4H), 7.79 (dd, J=1.5 Hz, J=8 Hz, 2H), 7.54 (dt, J=1.5 Hz, J=8 Hz, 2H), 7.50 (d, J=8 Hz, 2H), 7.46 (d, J=8 Hz, 2H), 7.32 (dt, J=1.5 Hz, J=8 Hz, 2H), 4.35 (t, J=8 Hz, 4H), 1.95-1.85 (m, 4H), 1.50-1.20 (m, 20H), 0.92 (t, J=6.5 Hz, 6H).

¹³C NMR (CDCl₃): δ165, 145, 141, 140, 131, 127, 127, 126, 125, 123, 123 ,122, 120, 119, 119, 109, 109, 43, 32, 29, 29, 29, 27, 23, 14.

### SYNTHETIC EXAMPLE 21

Using methods analogous to those used in synthetic example 20.

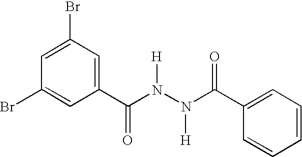

### 1-benzoyl-2-(3,5-dibromobenzoyl)hydrazine

¹H NMR (DMSO-d₆): δ10.7 (br s, 2H), 8.11 (s, 3H), 7.92 (d, J=8 Hz, 2H), 7.62 (t, J=8 Hz, 1H), 7.54 (t, J=8Hz, 2H).

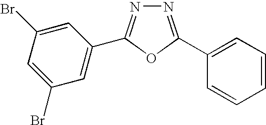

### 2-phenyl-5-(3,5-dibromophenyl)-1,3,4-oxadiazole

¹H NMR (CDCl₃): δ8.23 (d, J=1.5 Hz, 2H), 8.18 (dd, J=1.5 Hz, J=8 Hz, 2H), 7.88 (t, J=1.5 Hz, 1H), 7.65-7.56 (m, 3H).

¹³C NMR (CDCl₃): δ165, 162, 137, 132, 129, 128, 127, 127, 124, 123.

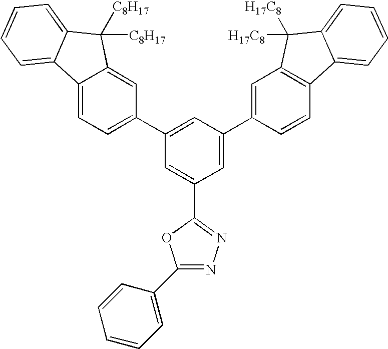

### 2-phenyl-5-(3,5-bis[9,9-bisoctylfluoren-2-yl]phenyl)-1,3,4-oxadiazole

¹H NMR (CDCl₃): δ8.50 (d, J=1.5 Hz, 2H), 8.28 (dd, J=1.5 Hz, J=8 Hz, 2H), 8.18 (t, J=1.5 Hz, 1H), 7.92 (d, J=8 Hz, 2H), 7.85-7.78 (m, 6H), 7.65-7.58 (m, 3H), 7.48-7.38 (m, 6H), 2.15-2.05 (m, 8H), 1.35-1.05 (m, 30H), 0.82 (t, J=6.5 Hz, 12H, 0.80-0.70 (m, 8H).

¹³C NMR (CDCl₃): δ165, 165, 152, 151, 143, 141, 140, 139, 132, 129, 129, 127, 127, 127, 126, 125, 124, 124, 123, 122, 120, 120, 55, 40, 32, 30, 29, 24, 23, 14.

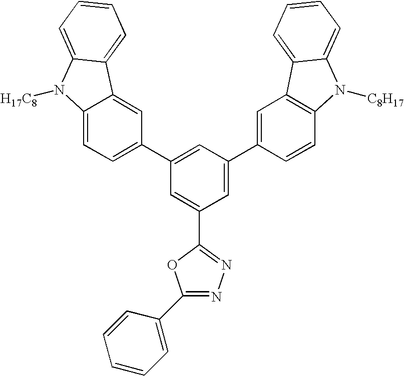

### 2-phenyl-5-(3,5-bis[9-octylcarbazol-3-yl]phenyl)-1,3,4-oxadiazole

¹H NMR (CDCl₃): δ8.54 (d, J=1.5 Hz, 2H), 8.47 (d, J=1.5 Hz, 2H), 8.29-8.24 (m, 5H), 7.92 (dd, J=1.5 Hz, J=8 Hz, 2H), 7.64-7.48 (m, 9H), 7.34 (dt, J=1.5 Hz, J=8 Hz, 2H), 4.38 (t, J=8 Hz, 4H), 2.05-1.90 (m, 41), 1.55-1.25 (m, 20H), 0.95 (t, J=6.5 Hz, 6H).

¹³C NMR (CDCl₃): δ165, 165, 144, 141, 140, 132, 131, 130, 129, 127, 126, 125, 125, 124, 124, 123, 123, 121, 119, 119, 109, 109, 43, 32, 29, 29, 29, 27, 23, 14.

### SYNTHETIC EXAMPLE 22

A range of terpolymers according to the formula TP 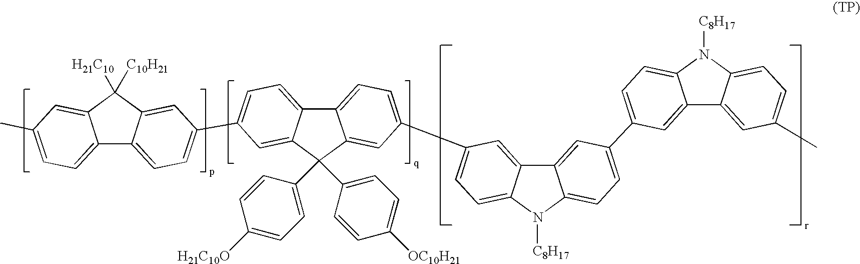
was prepared wherein the indices p, q and r indicate the percentage of the structural unit present in the terpolymer, C₈ is n-octyl and C₁₀ is 3,7-dimethyloctyl. Generally, the polymerization was performed as follows:

A flask containing a mixture of 4*q/100 mmol 2,7-bis(4,4,5,5-tetramethyl-1,3,2-dioxaborolyl)-9,9-bis(3,7-dimethyloctyl)fluorene, 4*p/100 mmol 2,7-dibromo-9,9-bis(3,7-dimethyloctyl)fluorene, 4*r/100 mmol bis[6-bromo-9-octylcarbazol-3-yl] (nk243), 2 drops of the phase transfer catalyst methyltrioctylammonium chloride (available from Aldrich, tradename aliquat 336), 20 ml (2 M) K₂CO₃ (aq) and 40 ml toluene was evacuated and charged with argon for three times, after which 2 mol % Pd(PPh₃)₄ was added. Evacuation and filling with argon was repeated once more and the mixture was stirred for 48 hours at 55° C. The polymers were end-capped by addition of 1.5 ml phenylboronic ester and some fresh catalyst, after which stirring at 55° C. was continued for 24 hours. The reaction mixture was added to 2 w % NaCN (aq) and stirred during several hours. This procedure was repeated once with fresh NaCN (aq). The organic layer was separated, dried (MgSO₄), filtered and concentrated. After fractionation and precipitation in methanol (twice) about 55% of polymer was obtained as white fibres.

Specific polymers obtained using the general procedure:

nk257: p=40; q=50; r=10.

M_{w}=23,000 and dispersion D=2.03, determined by gel permeation chromatography against a poly-styrene standard. Absorption spectrum λₘₐₓ=380 nm and fluorescence spectrum λₘₐₓ=430 nm with a shoulder at 450 nm.

nk277: p=30; q=50; r=20.

M_{w}=16,708 and dispersion D=2.79, determined by gel permeation chromatography against a poly-styrene standard. Absorption spectrum λₘₐₓ=380 nm and fluorescence spectrum λₘₐₓ=430 nm with a shoulder at 450 nm.

nk286: p=35; q=50; r=15.

M_{w}=11,865 and dispersion D=2.15, determined by gel permeation chromatography against a poly-styrene standard. Absorption spectrum λₘₐₓ=380 nm and fluorescence spectrum λₘₐₓ=430 nm with a shoulder at 450 nm.

nk287: p=25; q=50; r=25.

M_{w}=15,285 and dispersion D=2.46, determined by gel permeation chromatography against a poly-styrene standard. Absorption spectrum λₘₐₓ=380 nm and fluorescence spectrum λₘₐₓ=430 nm with a shoulder at 450 nm.

nk380: p=0; q=50; r=50.

### SYNTHETIC EXAMPLE 23

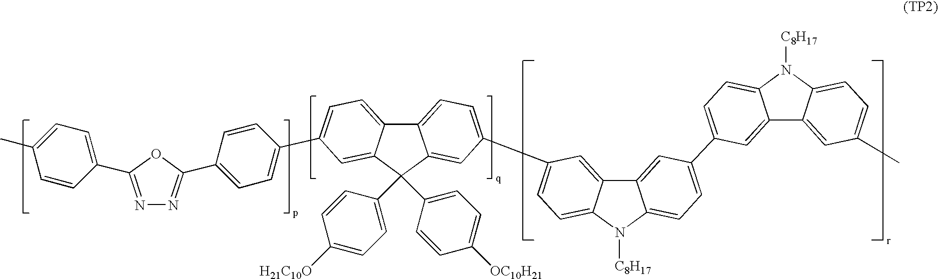

The terpolymer NK423 of the formula TP2, wherein p=20, q=50, and r=30 was synthesized in accordance with the procedure of synthetic example 22 except that instead of 2,7-dibromo-9,9-bis(3,7-dimethyloctyl)fluorene the dibromide 2,5-bis(4-bromophenyl)-1,3,4-oxadiazole is used.

Analogously, polymers of formula TP3 are prepared using 2-phenyl-5-(3,5-dibromophenyl)-1,3,4-oxadiazole.

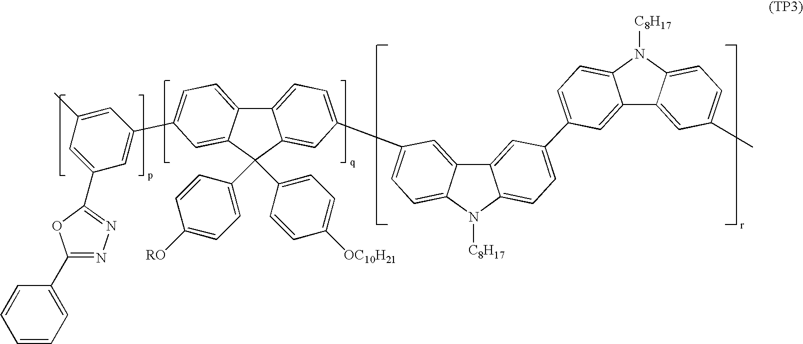

jjb857: p=20; q=50; r=30

nk477: p=50; q=0; r=50

### Embodiment 1

Of a number of carbazole compounds synthesized hereinabove, some in accordance with the invention and some not in accordance with the invention, a cyclovoltammogram is recorded. Specifically, cyclovoltammograms were recorded with 0.1 M tetrabutylammonium hexafluorophosphate as supporting electrolyte. The working electrode was a platinum disc (0.2 cm²), the counter electrode was a platinum plate (0.5 cm²), and a saturated calomel electrode was used as reference electrode, calibrated against Fc/Fc⁺ couple.

E⁰ is used for irreversible oxidations and reductions. It represents the peak potentials of the first oxidation wave or first reduction wave, depending on the subscript. For reversible (or quasi-reversible) oxidations and reductions E_{1/2} and ΔE are used. The cathodic and anodic waves are separated by ΔE, while the wave position is centered at E_{1/2}.

From the cyclovoltammograms so recorded, the half-wave oxidation potential(s) E_{1/2, ox} (in V), the ΔEₒₓ of the oxidation wave(s) (in mV) and the energy of the highest occupied molecular orbital (HOMO), E_{HOMO} (in eV) are derived. The energy of the HOMO is related to the an half-wave potential as E_{HOMO}=4.36+E_{1/2}. These data are collected in Table 1.

TABLE 1

| | carbazole | | | |
|---|---|---|---|---|
| compound | multimer | E_{1/2, ox} (V) | ΔEₒₓ (mV) | E_{HOMO} (eV) |
| nk202 | no | 1.45 | 163 | 5.81 |
| nk25320/ | no | 1.04 | 210 | 5.40 |
| nk25321 | | 1.46 | 210 | |
| | | 1.77 | 210 | |
| nk243 | yes | 0.99 | 135 | 5.35 |
| | | 1.36 | 125 | |
| nk30006 | yes | 0.91 | 86 | 5.27 |
| | | 1.16 | 103 | |
| | | 1.55 | 119 | |
| nk26803 | no | 1.40 | 204 | 5.76 |
| nk27003 | yes | 1.05 | 126 | 5.41 |
| | | 1.32 | 126 | |
| nk27206 | yes | 0.97 | 70 | 5.33 |
| | | 1.16 | 81 | |
| | | 1.53 | 97 | |
| nk29308 | yes | 0.83 | 50 | 5.19 |
| | | 0.93 | 29 | |
| | | 1.06 | 87 | |

None of the compounds in Table 1 show a reduction wave.

Table 1 clearly shows that the carbazole compounds which have carbazole multimers in accordance with the invention (entry in carbazole multimer column “yes”) have a HOMO energy in the range about 5.2 to about 5.4 eV. This range of HOMO energies is comparable to that observed in polyphenylenevinylene compounds which are known in the art (see eg Ho et al in Nature, 404, page 481, 2000) for having the capability of providing excellent hole-injecting contacts with high work-function electrode materials such as in particular indium tin oxide.

Table 1 further clearly shows that the HOMO energy of compounds having a monomer carbazole unit, which are not in accordance with the invention, is significantly larger, that is about 5.8 eV. Note that the HOMO energy for nk25320/25321 of 5.40 eV is attributable to the HOMO of the 9,9-bisdecyl-fluorene unit

### Embodiment 2

To further demonstrate the favorable hole-injecting properties of the carbazole multimer compounds of the present invention, the polymers synthesized in synthetic example 22 are used to manufacture a number of organic electroluminescent devices. The use of carbazole compounds in accordance with the invention excepted, the electroluminescent devices and their method of manufacturing is entirely conventional. The electroluminescent device is a layer stack ITO/PEDOT:PSS/nk257/BaAl, wherein ITO is an indiumtinoxide hole-injecting electrode, PEDOT:PSS is a hole-transport layer of poly-styrenesulphonic acid (PSS) doped poly-ethylenedioxythiophene (PEDOT) as available from Bayer AG or HC Starck, nk257 is an electroluminescent layer comprising a terpolymer labeled nk257 as synthesized in synthetic example 22 and BaAl is an electron-injecting electrode layer of a Ba layer and an Al layer. The electroluminescent device is connected to a voltage source and a voltage impressed. A photodiode is arranged on the light-emitting side of the electroluminescent device. The photo-diode current is a measure the amount of light emitted by the device. The photo-diode current is measured as a function of the voltage impressed. The result is plotted in FIG. 1.

Referring to FIG. 1, the electroluminescent device begins to emit light at about 4.5 V. Calibration of the diode photo-diode results teaches that at 4.5 V the brightness is in the range of 1 to 10 Cd/m². A diode current measured at about 6 V corresponds to a brightness of about 500 Cd/m².

The external efficiency of the device at 6 V is about 0.3 Cd/A.

FIG. 2, curve A, shows the electroluminescence spectrum of the device of FIG. 1. Light emission peaks at about 420 nm with a shoulder at 450 nm. Accordingly, the color of the light emitted is blue.

Similar results are obtained with devices comprising the terpolymers in accordance with the invention nk277, nk286 and nk287.

### Embodiment 3 (Not in Accordance with the Invention)

A comparative electroluminescent device not in accordance with the invention having, instead of an emissive layer of the terpolymeric carbazole compound in accordance with the invention of Embodiment 2, an emissive layer of the structurally similar polyvinylcarbazole is manufactured and measured in a manner analogous to that of Embodiment 2. Polyvinylcarbazole is a polymer comprising a monomer carbazole unit. To observe light emission in excess of 1 Cd/m2 a voltages in excess of 16 V is required. The corresponding efficiency is orders of magnitude lower than that of the device of the embodiment 2.

The results of Embodiments 2 and 3, in particular the low on-set for light emission, demonstrate the favorable charge-injecting properties of the carbazole compounds in accordance with the invention and their capability to electroluminesce blue light

### Embodiment 4

An electroluminescent device of the type described embodiment 2 is manufactured with the difference that the electroluminescent layer now includes a combination of the carbazole compound in accordance with the invention and a triplet emitter compound. The carbazole compound in accordance with the invention is the carbazole polymer nk257 and the triplet emitter is the orange-light emitting compound Irpq. Irpq is short for iridium(II) bis(2-phenylquinolyl-N,C^{2′}) acetylacetonate, is disclosed in Lamansky et al in, J. Am. Chem. Soc. 123 (2001) 4304 and used in a concentration of 6% by weight.

FIG. 2, curve C, shows the electroluminescence spectrum of this electroluminescent device. The device emits the orange light characteristic of the emission of Irpq. No blue emission characteristic of the carbazole compound (compare curve A) is observed. The measured device current versus drive voltage curve is substantially the same as that of the device in which the triplet emitter is absent but otherwise identical demonstrating that the charge-injection and transport is essentially handled by the carbazole host compound. Therefore, the carbazole host compound is energized by injection of holes and electrons and the energy thus stored on the host is subsequently transferred to the triplet emitter guest Irpq. The efficiency of the electroluminescent device is measured to be about 3 Cd/A at about 400 Cd/m², which is ten times higher than the device without the triplet emitter of Embodiment 2.

A further electroluminescent device is manufactured which is identical to the previous one except that the emissive layer comprises a guest-host system of 94% by weight of carbazole polymer nk257 as the host and 6% by weight of the the green-light-emitting triplet emitter Ir(ppy)₃ as the guest, where Ir(ppy)₃ is short for fac tris(2-phenylpyridine) iridium. Ir(ppy)₃ is available from American Dye Source Inc.

FIG. 2, curve B, shows the electroluminescence spectrum of this electroluminescent device. The device substantially emits the green light characteristic of the emission of Ir(ppy)₃. The device current versus drive voltage curve is substantially the same as that of the device of embodiment 2 which does not have the triplet emitter but is otherwise identical. Apparently, charge injection and transport is substantially handled by the carbazole host and the energy associated with the injection of charges (holes and electrons) is substantially transferred from the host to the guest.

FIG. 2, curve B shows that a small amount of the emission originates from the carbazole polymer. This indicates that not all energy (excitons) is transferred to the triplet emitter and/or that energy (charges or excitons) accepted by the triplet emitter is transferred back to the carbazole polymer. In either case, since light emission is most efficient from the triplet emitter, the efficiency of the electroluminescent device is expected to be adversely affected. Indeed, the efficiency is measured to be 0.3 Cd/A in the range from 50-400 cd/m2 which is significantly lower than the efficiency of the device with the orange triplet emitter Irpq where emission from the carbazole was not observed. A further factor which may adversely affect the efficiency of the electroluminescent device is the back-transfer of (excitonic) energy from the triplet emitter into the triplet state of the carbazole host polymer. Such back-transfer requires a triplet level lower in energy than the emission level of the triplet emitter.

### Embodiment 5

A series of solutions of carbazole compounds in accordance with the invention is prepared by dissolving an appropriate amount of the carbazole compound in methyl-THF and cooling the solution with liquid nitrogen to about 76 K to form a solid glass. At that temperature gated and un-gated photo-emission spectra are taken of the glass to distinguish between fluorescence (fast process) and phosphorescence (slow process). The phosphorescence spectra clearly show a series of peaks characteristic of vibronic progression. The highest energy peak of the lowest energy peak in the spectrum which can be assigned to a phosphorescent emission is taken to correspond to the triplet energy level of the carbazole compound. Triplet energy levels thus determined are collected in Table 2 below. FIG. 3 shows the corresponding spectrum.

TABLE 2

| | | Peak wavelength | |
|---|---|---|---|
| | type of | of phosphorescence | Triplet energy |
| Carbazole | multimer | [nm] | [cm-1] |
| Jjb790-04k | dimer | 451 | 22200 |
| (synthetic example 15) | | | |
| Jjb796-04k | dimer | 451 | 22200 |
| (synthetic example 3) | | | |
| Jjb800-05 | trimer | 454 | 22000 |
| (synthetic example 13) | | | |
| Nk303-05 | trimer | 454 | 22000 |
| (synthetic example 7) | | | |

Referring to Table 2, the triplet energy among the dimers is the same. The same is true for the trimers indicating that the substituent on the nitrogen atom has no influence on the triplet energy level. Further, the triplet energy of the dimers and trimers are almost the same indicating that triplet energy is substantially independent of the number of monomers in the multimer. The peak wavelength being at 450 nm, the triplet energy is sufficiently high to enable transfer energy to the orange Irpq and green Ir(ppy)₃ triplet emitter and prevent back-transfer to the triplet level. For comparison, the triplet energy of a carbazole monomer is about 24,600 cm−1. Apparently, the triplet state is of a rather localized character extending across about two carbazole units.

### Embodiment 6

On an ITO-covered glass substrate, a layer stack HTL/LEL/HBL/ETL/EIE is deposited by means of vacuum deposition having the following composition: 30.1 nm α-NPD/30 nm (91.7% wt Jjb796-04k, 8.3% wt Ir(ppy)₃)/10 nm bathocuproin/40 mn Alq₃/1.5 nm Li-benzoate/70 nm Al wherein α-NPD N,N′-di(naphtalen-1-yl)-N,N′-diphenyl-benzidine and bathocuproin is 2,9-dimethyl-4,7-diphenyl-1,10-phenantroline. Alq₃ is aluminum trisoxine. The carbazole dimer Jjb796-04k is evaporated at 240° C. The device emits green light characteristic of the triplet emitter Ir(ppy)₃). The external efficiency of the device is about 30 to 35 Cd/A. A device having 15 to 25 Cd/A external efficiency is obtained if the dimer carbazole Jjb796-04k is replaced with the carbazole trimer Jjb800-05.

Although not necessarily wishing to be bound by any theory, it is believed that such efficiencies can only be obtained if triplet excitons on the carbazole compound are efficiently transferred to the triplet emitter and/or triplet excitons on the triplet emitter are effectively prevented from being transferred to the carbazole polymer. Such efficient transfer and/or effective prevention of back-transfer requires the triplet of the carbazole compound to be located above the triplet level of the triplet emitter. According to Table 2 the triplet level of the carbazole dimer and triier is about 22,00 cm−1 and the triplet level of Ir(ppy)₃ about 18,000 cm−1.

Further, the energy of the HOMO of the dimer JjB796-04k is 5.32 eV and of the trimer is 5.25 eV allowing facile hole injection.

### Embodiment 7

Analogous to embodiment 2 an electroluminescent device of the following layer stack is obtained: ITO/PEDOT:PSS/(x wt % nk380, 100−x wt % lrpq)/BaAl. More specifically, a series of devices is prepared in which the amount of Irpq is varied. Throughout the series, light emission characteristic of the orange triplet emitter Irpq is observed. The device with 100−x=8 wt % Irpq has the best performance, the efficiency being 12 Cd/A at about 15 to 20 V drive voltage.

### Embodiment 8

Analogous to the previous embodiment electroluminescent devices having the following stack of layers ITO/PEDOT:PSS/LEL/BaAl are manufactured and the efficiency of each is measured. The results can be summarized as follows:

LEL=light emissive layer of 92 wt % nk432 and 8 wt % IRA where IRA is a red-emitting triplet emitter. Efficiency is about 5 Cd/A at 5 V.

LEL=light emissive layer of 92 wt % jjb857 and 8 wt % IRA where IRA is a red-emitting triplet emitter. Efficiency is about 7.3 Cd/A at 6 V.

LEL=light emissive layer of 92 wt % nk432 and 8 wt % the green light-emitting triplet emitter Ir(ppy)₃. Efficiency is about 2 Cd/A at 5 V.

LEL=light emissive layer of 92 wt % JJB857 and 8 wt % of the green light-emitting triplet emitter Ir(Ppy)₃. Efficiency is about 10 Cd/A at 7 V.

In all instances light emission characteristic of the triplet emitter is obtained.

A particularly efficient EL device is obtained with a light-emissive layer of 92 % wt of nk477 and 8% wt of Ir(ppy)₃. Efficiency is measure to be about 16.8 Cd/A at 6.8 V.

The efficiency is obtained in a range brightness range of about 1000-6000 cd/m2. The emission of the device corresponds to that of the triplet emitter. No rest emission of the carbazole polymer is observed.

1. A carbazole compound comprising a carbazole multimer unit of formula (I) 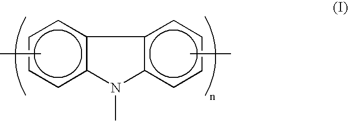
wherein each carbazole unit may be unsubstituted or substituted with one or more substituents and n is larger than or equal to 2.

2. A carbazole compound as claimed in claim 1 wherein the carbazole multimer unit includes a 2,2′-bicarbazole-diyl unit.

3. A carbazole compound as claimed in claim 1 wherein the carbazole multimer unit includes a 3,3′-bicarbazole-diyl unit.

4. A carbazole compound as claimed in claim 1, 2 or 3 wherein the carbazole compound is a polymer having a repeating unit comprising a carbazole multimer unit of the formula (X) 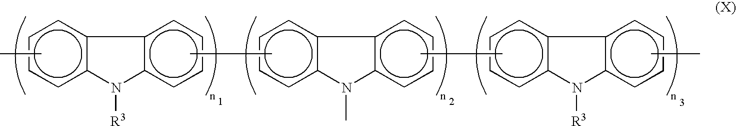
wherein each carbazole unit may be unsubstituted or substituted with one or more substituents and R³ is, the same or different at each occurrence, an alkyl, heteroalkyl, aryl or heteroaryl substituent having not more than 40 non-hydrogen atoms;
n₁ and n₃ are whole numbers including 0, n₂ is 0 or 1 and (n₁+n₂+n₃)≧2.

5. A combination of a carbazole compound as claimed in claim 1, 2, 3 or 4 and a light-emissive compound capable of accepting energy from the carbazole compound.

6. A combination as claimed in claim 5 wherein the light-emissive compound is a triplet emitter compound.

7. A combination of a charge-transporting conjugated compound having a lowest triplet level with an energy of about 21,000 cm−1 or higher and a triplet emitter compound having an emission level with an energy of about 21,000 cm−1 or lower.

8. A combination of a charge-transporting conjugated compound having a lowest triplet level with an energy of about 22,000 cm−1 or higher and a triplet emitter compound having an emission level with an energy of about 22,000 cm−1 or lower.

9. A combination as claimed in claim 7 or 8 wherein the compound is a polymer.

10. A combination as claimed in claim 6, 7, 8 or 9 wherein the compound or polymer is a compound or polymer as claimed in any one of the claims 1, 2, 3 or 4.

11. A combination as claimed in claim 7 or 8 wherein the highest occupied molecular orbital of the charge-transporting conjugated compound has an energy of less than or equal to about 5.4 eV.

12. A combination as claimed in claim 7 or 8 wherein the highest occupied molecular orbital of the charge-transporting conjugated compound has an energy of less than or equal to about 5.3 eV.

13. An electroluminescent device including a carbazole compound as claimed in any one of the claims 1, 2, 3 or 4 or a combination as claimed in claim 5, 6, 7, 8, 9 10, 11 or 12.
